(19) United States
(12) Patent Application Publication
(10) Pub. No.: US 2014/0031712 A1
(43) Pub. Date: Jan. 30, 2014

(54) SYSTEM AND METHOD FOR DETECTING NEUROLOGICAL DETERIORATION

(71) Applicants: Moshe Herskovitz, Haifa (IL); Goded Shahaf, Haifa (IL)

(72) Inventors: Moshe Herskovitz, Haifa (IL); Goded Shahaf, Haifa (IL)

(73) Assignee: NEUROKEEPER TECHNOLOGIES LTD., Netanya (IL)

(21) Appl. No.: 14/110,264

(22) PCT Filed: Apr. 5, 2012
(86) PCT No.: PCT/IL12/50132

§ 371 Date: Oct. 7, 2013

Related U.S. Application Data
(60) Provisional application No. 61/471,735, filed on Apr. 5, 2011.

(51) Int. Cl. A61B 5/0482; A61B 5/00; A61B 5/0484; A61B 5/04

(52) U.S. Cl. CPC A61B 5/0482; A61B 5/04012; A61B 5/0006; A61B 5/0022; A61B 5/7405; A61B 5/742; A61B 5/4812; A61B 5/0484
USPC 600/545; 600/544

(57) ABSTRACT

A system for detecting a neurological deterioration in a patient, the system constituted of: an analyzing module; and at least one input port arranged to receive a plurality of first samples of electroencephalographic (EEG) activity of the patient and a plurality of second samples of EEG activity of the patient and further arranged to output the received samples to the analyzing module, wherein the analyzing module is arranged to: compare a function of the received first EEG samples to a function of the received second EEG samples; determine the difference between the outcome of the comparison and a baseline value; and output a neurological deterioration signal responsive to the determined difference.

### CROSS-REFERENCE TO RELATED APPLICATIONS

This application claims priority from U.S. Provisional Patent Application Ser. No. 61/471,735, filed Apr. 5, 2011, entitled “ELECTROENCEPHALOGRAPHIC ANALYTICAL SYSTEMS AND METHODS”, the entire contents of which are incorporated herein by reference.

### TECHNICAL FIELD

The present invention relates generally to the field of electroencephalographic (EEG) signal analyses, and in particular to detecting neurological deterioration responsive to the analyses.

### BACKGROUND

A cerebrovascular accident (CVA), commonly called a stroke, is the rapid loss of brain function due to disturbance in the blood supply to the brain. EEG signals are in some cases used as a method for identification of a CVA. The EEG signals are typically analyzed in a plurality of frequency bands, namely Delta (0-3 Hz), Theta (4-7 Hz), Alpha (8-12Hz) and Beta (13-30 Hz). Several characteristic changes are reported in the EEG signal immediately following CVA onset, in the timescale of seconds to minutes. Examples of these changes include: attenuation of activities, particularly in the beta and alpha frequencies, hereinafter described as the first differential diagnosis factor and denoted as DDF1; regional attenuation without delta (RAWOD), hereinafter described as the second differential diagnosis factor and denoted DDF2; enhancement of slower activities, e.g. delta, hereinafter described as the third differential diagnosis factor and denoted DDF3; and reduction and/or stagnation in the variability of the EEG signal, hereinafter described as the fourth differential diagnosis factor and denoted DDF4.

During sleep, EEG signals typically change according to the various sleep stages, as outlined below. In sleep stage 1, non-rapid eye movement (NREM-I) is characterized by slow rolling eye movements (SREMs), hereinafter termed sleep characteristic 1 and denoted SH1, which are usually the first evidence of drowsiness seen on the EEG. SREMs of drowsiness are most often horizontal but can be vertical or oblique and their distribution is similar to eye movements in general. SREMs are typically slow, e.g. 0.25-0.5Hz. SREMs disappear in NREM-II and in deeper sleep stages, as will be discussed further below.

Stage 1 is further characterized by attenuation of the alpha rhythm, hereinafter defined as sleep characteristic 2 and denoted SH2. Attenuation of the alpha activity typically occurs together with, or at a small time difference from, an SREM. The alpha rhythm gradually becomes slower, less prominent and fragmented. Central or frontocentral theta activity exhibit alterations, hereinafter defined as sleep characteristic 3 and denoted SH3. Enhanced beta activity, hereinafter defined as sleep characteristic 4 and denoted SH4 is further observed.

Positive occipital sharp transients of sleep (POSTS), hereinafter defined as sleep characteristic 5 and denoted SH5, have a positive maximum at the occipital lobes, are contoured sharply and occur in early sleep, i.e. in sleep stages I and II, to be discussed further below. POSTS's morphology is classically described as a reverse check mark with amplitude of about 50-100 μV. POSTS's typically occur in runs of 4-5 Hz and are bisynchronous, although they may be asymmetric. They persist in stage II sleep but usually disappear in subsequent stages.

Vertex sharp transients, otherwise called vertex waves or V-waves, are almost universally found in sleep stage 1, and are hereinafter defined as sleep characteristic 6, denoted SH6. A V-waves' amplitude is typically about 50-150 μV. V-waves can be contoured sharply and occur in repetitive runs, especially in children. V-waves persist in stage II sleep but usually disappear in subsequent stages.

Sleep stage 2 non-rapid eye movement (NREM-II) is characterized by sleep spindles, hereinafter defined as sleep characteristic 7 and denoted SH7, which have frequencies of about 12-16 Hz and typically 14 Hz, are typically maximally apparent at the central region of the brain, i.e. the vertex, although occasionally they may predominate in the frontal regions. Sleep spindles occur in short bursts of waxing and waning spindle-like (fusiform) rhythmic activity. The amplitude of sleep spindles is usually of about 20-100 μV. Extreme spindles are characterized by an unusually high-voltage amplitude of about 100-400 μV and prolonged (>20 seconds) spindles located over the frontal regions.

K-complexes, hereinafter defined as sleep characteristic 8 and denoted SH8, are characterized by an amplitude of >100 μV and breadth of >200 ms. K-complexes are diphasic, are of a transient shape and are often associated with sleep spindles. The location of K-complexes are at the frontocentral region, with a typical maximum at the midline. K-complexes occur spontaneously and are elicited as an arousal response. K-complexes may have an association with blood pressure fluctuation during sleep.

Sleep stage 3 exhibits non-rapid eye movement (NREM-III) characterized by delta sleep pattern, hereinafter defined as sleep characteristic 9 and denoted SH9, which is characterized by delta activity. Delta sleep pattern is typically generalized and polymorphic or semirhythmic. According to sleep staging criteria on polysomnography, delta sleep pattern stages are defined by the presence of delta activity for more than 20% of the time, often with an amplitude threshold of at least 75 μV. Sleep spindles and K-complexes, hereinafter defined as sleep characteristic 10 and denoted SH10, may persist in NREM-III but they are not as prominent.

Sleep stage 4 exhibits non-rapid eye movement (NREM-IV) characterized by delta activity is present during a greater portion of the time, typically more than 50%, and is hereinafter defined as sleep characteristic 11 and denoted SH11. A distinction between NREM-III and NREM-IV can be drawn on a quantitative basis, typically based on the amount of delta activity. NREM-III is defined by delta activity present during 20%-50% of the time, as opposed to the increased delta activity of NREM-IV.

Sleep spindles and K-complexes, hereinafter defined as sleep characteristic 12 and denoted SH12, may persist to some degree in NREM-IV but they are more attenuated and less common than as in NREM-III.

Sleep stage 5 exhibits rapid eye movements (REMs), hereinafter defined as sleep characteristic 13 and denoted SH13, which are typically manifest in EEG signals. Sleep stage 5 further exhibits muscle atonia, hereinafter defined as sleep characteristic 14 and denoted SH14, which may be manifest in EEG signals. Desynchronization in the

EEG signal is observed, hereinafter defined as sleep characteristic 15 and denoted SH15, particularly as compared to the EEG activity during NERM III or IV. The background EEG activity changes from that seen in NERM III or IV to faster and lower voltage activity (theta and beta), resembling wakefulness, hereinafter defined as sleep characteristic 16 and denoted SH16.

Sleep stage 5 further exhibits EEG characteristic saw tooth waves, hereinafter defined as sleep characteristic 17 and denoted SH17, which are a special type of central theta activity that has a notched morphology resembling the blade of a saw and usually occurs close to rapid eye movements (i.e. phasic REM).

Rapid treatment of CVA is critical, so as to minimize brain damage. However to accomplish treatment it is necessary to be aware of the existence of the CVA. One of the dangers of CVA during sleep is that it is not diagnosed until the patient wakes up. In some instances it is not diagnosed at all since the patient was not aware of the CVA occurrence during sleep, and thus does not report the incidence.

Unfortunately, the prior art methods and systems do not provide for CVA detection, or other neurological deteriorations such as sub-arachnoid hemorrhage and infection, during the various stages of sleep. There is thus a long felt need for a system arranged to detect neurological deterioration in a patient, while the patient is asleep.

### SUMMARY

Accordingly, it is a principal object to overcome at least some of the disadvantages of prior art systems for detecting neurological deterioration. In one embodiment, a system for detecting a neurological deterioration in a patient is provided, the system comprising: an analyzing module; and at least one of input port arranged to receive a plurality of first samples of electroencephalographic (EEG) activity of the patient and a plurality of second samples of EEG activity of the patient and further arranged to output the received samples to the analyzing module, wherein the analyzing module is arranged to: compare a function of the received first EEG samples to a function of the received second EEG samples; determine the difference between the outcome of the comparison and a baseline value; and output a neurological deterioration signal responsive to the determined difference.

In one embodiment, the received at least one first EEG sample is associated with a first cerebral hemisphere of the patient and the second plurality of received EEG samples are associated with a second cerebral hemisphere of the patient opposing the first cerebral hemisphere. In another embodiment, the comparison comprises determining the correlation between the function of the first plurality of received EEG samples and the function of the second plurality of received EEG samples.

In one embodiment, the at least one input port comprises a plurality of input ports, each in communication with a particular electrode arranged to sample EEG samples of the patient, and wherein the received plurality of first EEG samples comprise: a first set of first EEG samples received from a first of a pair of first electrodes; and a second set of first EEG samples received from a second of a pair of first electrodes, wherein the received plurality of second EEG samples comprise: a first set of second EEG samples received from a first of a pair of second electrodes; and a second set of second EEG samples received from a second of a pair of second electrodes, wherein the function of the received plurality of first EEG samples comprises the difference between the amplitudes of the first set of first EEG samples and the amplitudes of the second set of first EEG samples, and wherein the function of the received plurality of second EEG sample comprises the difference between the amplitudes of the first set of second EEG samples and the amplitudes of the second set of second EEG samples.

In another embodiment, the at least one input port comprises a plurality of input ports, each in communication with a particular electrode arranged to sample EEG samples of the patient, wherein the received plurality of first EEG samples comprise: a first set of first EEG samples received from a first of a pair of first electrodes; and a second set of first EEG samples received from a second of a pair of first electrodes, wherein the received plurality of second EEG samples comprise: a first set of second EEG samples received from a first of a pair of second electrodes; and a second set of second EEG samples received from a second of a pair of second electrodes, wherein the function of the received plurality of first EEG samples comprises the correlation between the first set of first EEG samples and the second set of first EEG samples, and wherein the function of the received plurality of second EEG samples comprises the correlation between the first plurality of second EEG samples and the second plurality of second EEG samples.

In one embodiment, the comparison comprises determining one of: an average of the amplitude values of the plurality of first EEG samples and the plurality of second EEG samples; the variability of the amplitude values of the plurality of first EEG samples and the plurality of second EEG samples; and the rate of change of the amplitude values of the plurality of first EEG samples and the plurality of second EEG samples. In another embodiment, the baseline value comprises one of: a general population baseline; and a baseline associated with the patient.

In one embodiment, the system further comprises: a patient stimulation device, in communication with the analyzing module, and arranged, responsive to a non-stimulated state patient stimulation signal, to provide a non-awakening stimulation to the patient, the non-awakening stimulation arranged to at least partially increase the EEG activity of the patient, wherein the analyzing module is further arranged to: compare the amplitude of each of the received EEG samples to a predetermined minimum value; and in the event the amplitude of one of the received EEG samples is less than the predetermined minimum value, output the patient stimulation signal. In one further embodiment, the patient stimulation device is arranged to provide awakening stimulation to the patient responsive to a stimulated state patient stimulation signal received from the analyzing module, wherein the awakening stimulation is arranged to awaken the patient.

In another embodiment, the system further comprises: a patient stimulation device, in communication with the analyzing module, and arranged, responsive to a non-stimulated state patient stimulation signal, to provide a non-awakening stimulation to the patient, wherein the non-awakening stimulation is arranged to at least partially increase the EEG activity of the patient, and wherein the non-stimulated state patient stimulation signal is responsive to the output neurological deterioration signal. In one further embodiment, the patient stimulation device is arranged to provide awakening stimulation to the patient responsive to an stimulated state patient stimulation signal received from the analyzing module, wherein the awakening stimulation is arranged to awaken the patient, and wherein the stimulated state patient stimulation signal is responsive to the output neurological deterioration signal.

In one embodiment, the analyzing module further comprises: a communication module arranged to output the neurological deterioration signal, wherein the communication module comprises one of: a connection to a an audible output device; a connection to a graphical output device; a connection to a short messaging service; a connection to a telephony network; a connection to the Internet; and a connection to a medical network. In another embodiment, each of the plurality of input ports is in communication with a particular electrode, the received plurality of first EEG samples being received from at least one first electrode and the received plurality of second EEG samples being received from at least one second electrode, wherein the analyzing module is further arranged to determine the standard deviation of the received EEG samples from each of the first and second electrodes over a first predetermined time period, and in the event the determined standard deviation for any the first and second electrodes is greater than a first predetermined value, the analyzing module is further arranged to output a noise detection signal for the particular electrode.

In one embodiment, the neurological deterioration signal is output in the event the determined difference is greater than a second predetermined value for a second predetermined time period. In another embodiment, the baseline value is selectable from a plurality of baseline values, wherein the analyzing module is further arranged to: determine the current sleep stage of the patient, responsive to the received plurality of first EEG samples and the plurality of second EEG samples; and select a particular baseline value responsive to the determined sleep stage of the patient.

Independently, a method of detecting a neurological deterioration in a patient is provided, the method comprising: receiving a plurality of first samples of electroencephalographic (EEG) activity of the patient and a plurality of second samples of

EEG activity of the patient; comparing a function of the received first EEG samples to a function of the received second EEG samples; determining the difference between the outcome of the comparison and a baseline value; and outputting a neurological deterioration signal responsive to the determined difference.

In one embodiment, the received first EEG samples are associated with a first cerebral hemisphere of the patient and the receive second EEG samples are associated with a second cerebral hemisphere of the patient opposing the first cerebral hemisphere. In another embodiment, the comparing comprises determining the correlation between the function of the received first EEG samples and the function of the received second EEG samples.

In one embodiment, the received plurality of first EEG samples comprise: a first set of first EEG samples received from a first of a pair of first electrodes; and a second set of first EEG samples received from a second of a pair of first electrodes, wherein the received plurality of second EEG samples comprise: a first set of second EEG samples received from a first of a pair of second electrodes; and a second set of second EEG samples received from a second of a pair of second electrodes, wherein the function of the received plurality of first EEG samples comprises the difference between the amplitudes of the first set of first EEG samples and the amplitudes of the second set of first EEG samples, and wherein the function of the received plurality of second EEG samples comprises the difference between the amplitudes of the first set of second EEG samples and the amplitudes of the second set of second EEG samples.

In another embodiment, the received plurality of first EEG samples comprise: a first set of first EEG samples received from a first of a pair of first electrodes; and a second set of first EEG samples received from a second of a pair of first electrodes, wherein the received plurality of second EEG samples comprise: a first set of second EEG samples received from a first of a pair of second electrodes; and a second set of second EEG samples received from a second of a pair of second electrodes, wherein the function of the received plurality of first EEG samples comprises the correlation between the first set of first EEG samples and the second set of first EEG samples, and wherein the function of the received plurality of second EEG samples comprises the correlation between the first set of second EEG samples and the second set of second EEG samples.

In one embodiment, the comparing comprises determining one of: an average of the amplitude values of the plurality of first EEG samples and the plurality second EEG samples; the variability of the amplitude values of the plurality of first EEG samples and the plurality of second EEG samples; and the rate of change of the amplitude values of the plurality of first EEG samples and the plurality of second EEG samples. In another embodiment, wherein the baseline value comprises one of: a general population baseline; and a baseline associated with the patient.

In one embodiment, the method further comprises: providing, responsive to a non-stimulated state patient stimulation signal, non-awakening stimulation to the patient, the non-awakening stimulation arranged to at least partially increase the EEG activity of the patient; comparing the amplitude of each of the received EEG samples to a predetermined minimum value; and in the event the amplitude of one of the received EEG samples is less than the predetermined minimum value, outputting the patient stimulation signal. In one further embodiment, the method further comprises: providing awakening stimulation to the patient responsive to a stimulated state patient stimulation signal, wherein the awakening stimulation is arranged to awaken the patient.

In another embodiment, the method further comprises: providing, responsive to a non-stimulated state patient stimulation signal, non-awakening stimulation to the patient, wherein the non-awakening stimulation is arranged to at least partially increase the EEG activity of the patient, and wherein the non-stimulated state patient stimulation signal is responsive to the output neurological deterioration signal. In one further embodiment, the method further comprises: providing awakening stimulation to the patient responsive to an stimulated state patient stimulation signal, wherein the awakening stimulation is arranged to awaken the patient, and wherein the stimulated state patient stimulation signal is responsive to the output neurological deterioration signal.

In one embodiment, the outputting the neurological deterioration signal comprises connecting to one of: an audible output device; a graphical output device; a short messaging service; a telephony network; the Internet; and a medical network. In another embodiment, the received plurality of first EEG samples are received from at least one first electrode and the received plurality of second EEG samples are received from at least one second electrode, the method further comprising: determining the standard deviation of the received EEG samples from each of the first and second electrodes over a first predetermined time period, and in the event the determined standard deviation for any of the first and second electrodes is greater than a first predetermined value, outputting a noise detection signal for the particular electrode.

In one embodiment, the neurological deterioration signal is output in the event the determined difference is greater than a second predetermined value for a second predetermined time period. In another embodiment, the baseline value is selectable from a plurality of baseline values, the method further comprising: determining the sleep stage of the patient responsive to the received plurality of first EEG samples and the received plurality of second EEG samples; and selecting a particular baseline value responsive to the determined sleep stage of the patient.

Additional features and advantages will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1A illustrates a high level schematic diagram of a system for detecting neurological deterioration in a patient, comprising an analyzing module in communication with a plurality of electrodes;

FIG. 1B illustrates a high level block diagram of the analyzing module of FIG. 1A;

FIG. 1C illustrates a high level flow chart of a first embodiment of a method of operation of the system of FIGS. 1A-1B;

FIG. 1D illustrates a high level flow chart of a second embodiment of a method of operation of the system of FIGS. 1A-1B;

FIG. 1E illustrates a high level flow chart of a third embodiment of a method of operation of the system of FIGS. 1A-1B;

FIG. 1F illustrates a high level flow chart of a fourth embodiment of a method of operation of the system of FIGS. 1A-1B;

FIG. 2A illustrates a plot of fronto-temporal EEG activity in the left hemisphere of a patient exhibiting a CVA in the left cerebral hemisphere and a plot of fronto-temporal activity in the right hemisphere of the patient;

FIG. 2B illustrates a cranial CT scan of the patient of FIG. 2A;

FIG. 2C illustrates a plot of fronto-temporal EEG activity in the left hemisphere of a healthy individual and a plot of fronto-temporal activity in the right hemisphere of the healthy individual;

FIG. 2D illustrates plots of a calculated parameter for the patient of FIG. 2A and the healthy individual of FIG. 2C;

FIGS. 3A-4B illustrate various graphs of the correlation between EEG signals of a plurality of electrodes, the EEG signals sampled from a plurality of patients suffering from CVA;

FIGS. 5A-5B illustrate various graphs of the correlation between EEG signals of a plurality of electrodes, the EEG signals sampled from a healthy individual;

FIG. 5C illustrates the amplitude of the received EEG signals of a plurality of electrodes, the EEG signals sampled from the healthy individual of FIGS. 5A-5B;

FIGS. 6A-6B illustrate various graphs of the correlation between EEG signals of a plurality of electrodes, the EEG signals sampled from a healthy individual;

FIG. 6C illustrates the amplitude of the received EEG signals of a plurality of electrodes, the EEG signals sampled from the patient of FIGS. 6A-6B;

FIG. 7A illustrates a graph of the amplitude of EEG signals received from two electrodes;

FIG. 7B illustrates a graph of the differences of the amplitudes of EEG signals received from two pairs of electrodes;

FIG. 7C illustrates a graph of the correlation of EEG signals received from two pairs of electrodes;

FIG. 8A illustrates a graph of the correlation of the differences of EEG signals received from a plurality of pairs of electrodes, the EEG signals sampled from a third patient suffering from CVA;

FIG. 8B illustrates a graph of the correlation of the differences of EEG signals received from a plurality of pairs of electrodes, the EEG signals sampled from a second patient suffering from CVA;

FIG. 8C illustrates a graph of the correlation of the differences of EEG signals received from a plurality of pairs of electrodes, the EEG signals sampled from a healthy individual;

FIG. 9 illustrates a graph of the lowest correlation average between pairs of electrodes for each of a plurality of patients suffering from CVA and a plurality of healthy individuals; and

FIGS. 10A-10C illustrate graphs of the noise in EEG signals received from a plurality of electrodes.

### DETAILED DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

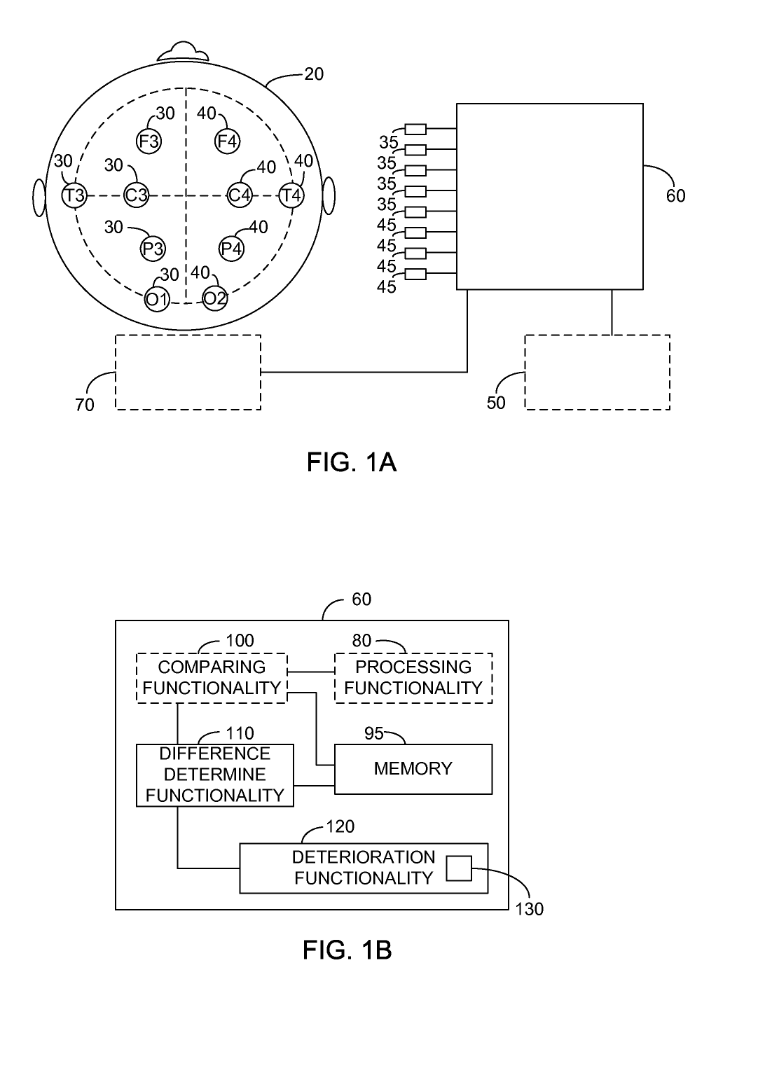

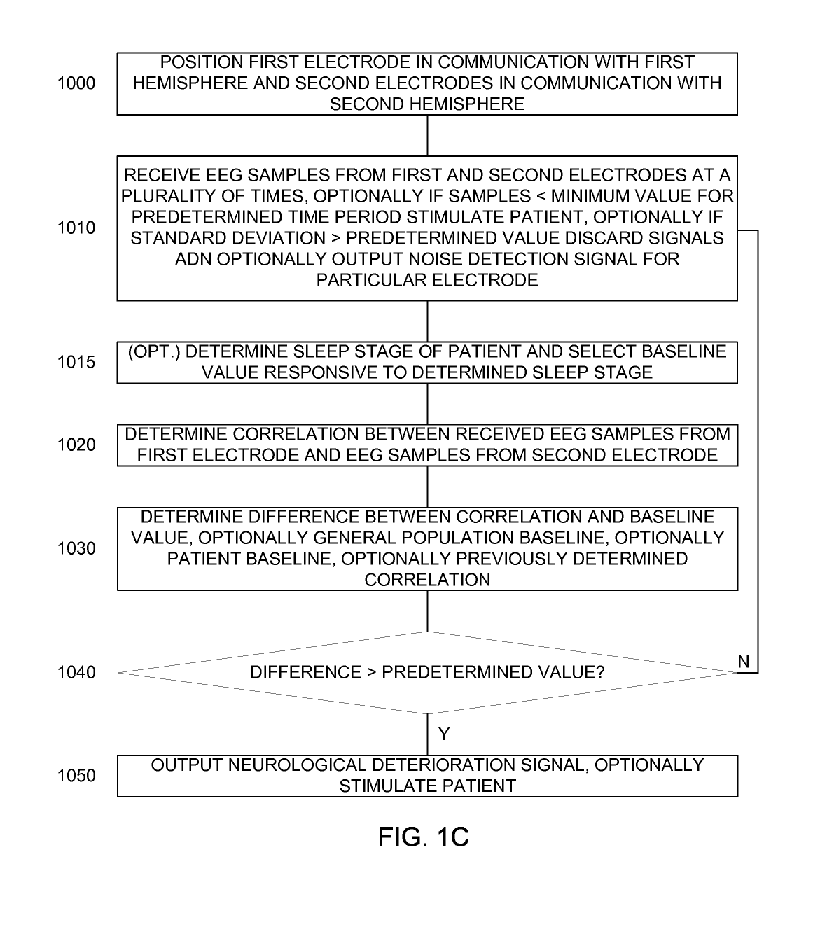

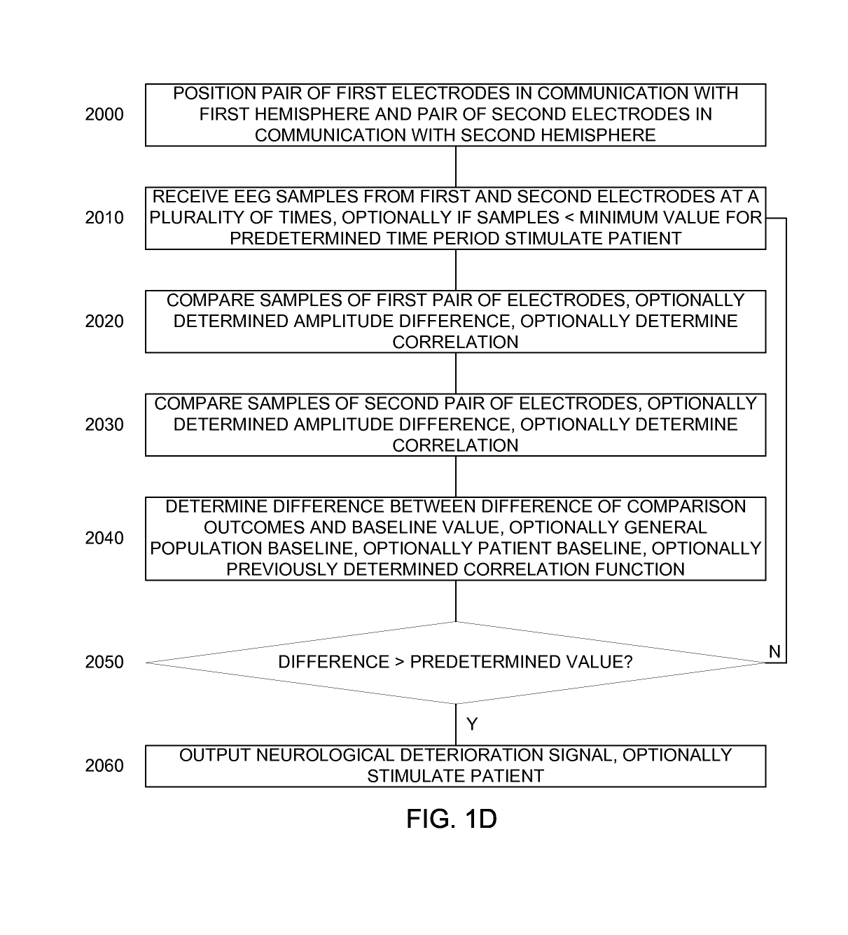

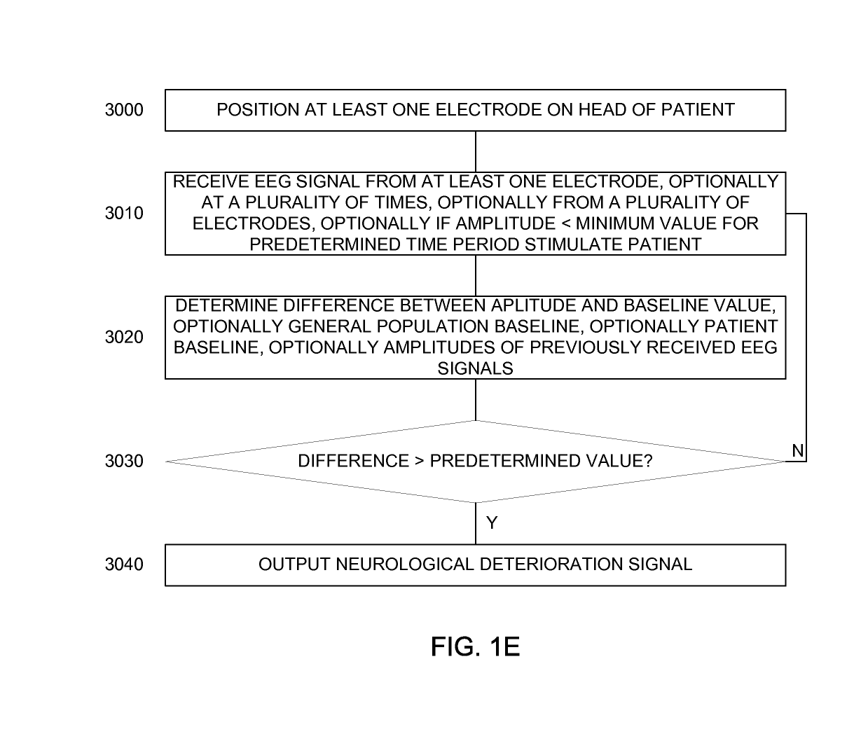

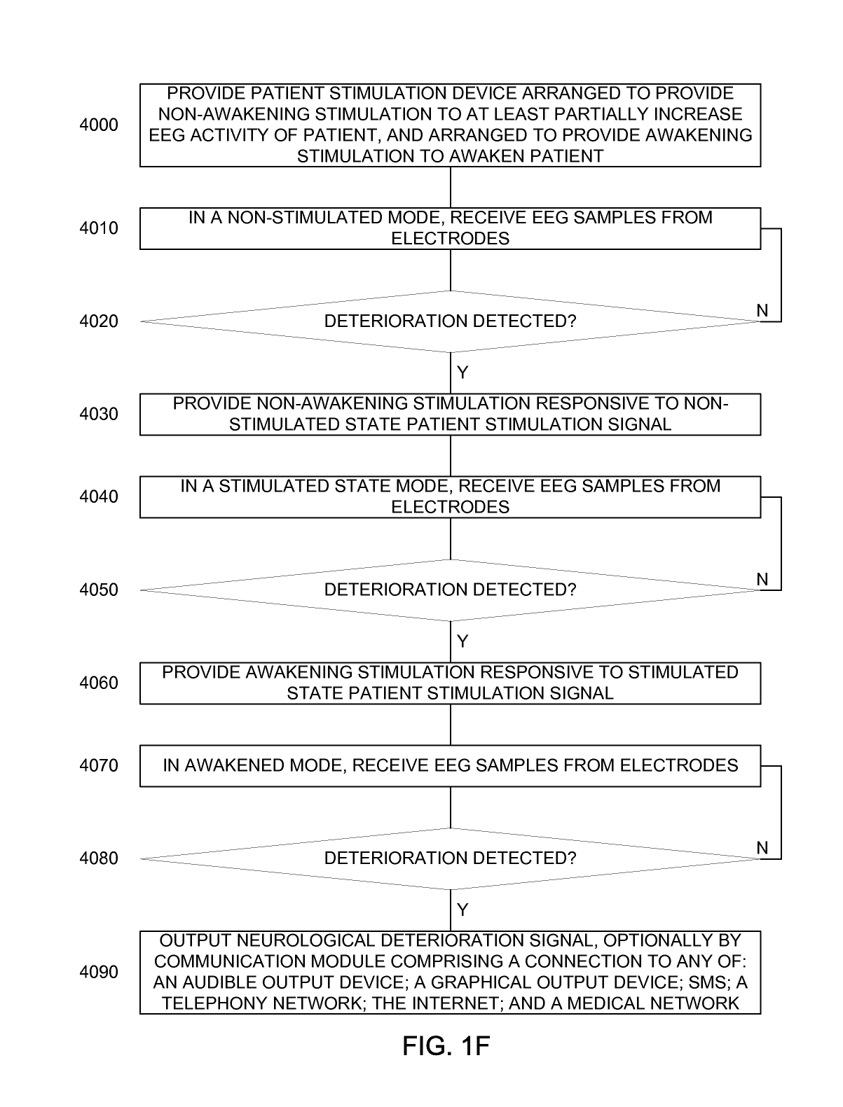

FIG. 1A illustrates a high level schematic diagram of a system 10 for detecting neurological deterioration in a patient 20, system 10 comprising: a plurality of electrodes 30; a plurality of input ports 35; a plurality of electrodes 40; a plurality of input ports 45; an optional user interface 50; an analyzing module 60; and an optional patient stimulation module 70. Electrodes 30 and electrodes 40 are arranged to be attached to the head of patient 20 by any of a plurality of attachment devices, such as an adhesive, a stretchable elastic band or EEG headwear known to the prior art, without limitation. In one embodiment, each electrode 30, 40 further comprises an indicator (not shown), such as an LED, arranged to indicate proper operation and/or connection and/or positioning of the electrode.

Electrodes 30 are each in communication with analyzing module 60 via respective input ports 35 and electrodes 40 are each in communication with analyzing module 60 via respective input ports 45. In one embodiment, each electrode 30, 40 comprises a portable power source (not shown) and a wireless transmitter (not shown), such as: an infrared transmitter; a wireless personal area network (WPAN) transmitter; a wireless local area network (WLAN) transmitter; or a wide area network (WAN) transmitter, without limitation. The aforementioned wireless transmitters are typically coupled to portable energy sources, e.g. electrical batteries. In such an embodiment, input ports 35 and 45 are in communication with a wireless receiver (not shown) arranged to receive signals from the various electrodes 30, 40. In one non-limiting embodiment, a plurality of wireless receivers are provided (not shown) each associated with a particular electrode 30, 40 and arranged to receive signals therefrom, each input port 35, 45 in communication with a particular one of the wireless receivers.

Electrodes 30 and electrodes 40 are each arranged to receive samples of EEG activity from patient 20. In particular, electrodes 30 are arranged to be disposed along the left side of the head of patient 20 and to receive samples of EEG activity from the left cerebral hemisphere of patient 20, and electrodes 40 are arranged to be disposed along the right side of the head of patient 20 and to receive samples of EEG activity from the right cerebral hemisphere of patient 20. A preferable example of the division of a human brain is in accordance with the international 10-20 system, which is an internationally recognized method to describe and apply the location of scalp electrodes, widely accepted in the art of electroencephalography. Each site has a letter to identify the lobe and a number to identify the hemisphere location. The letters F, T, C, P and O respectively represent the Frontal, Temporal, Central, Parietal and Occipital regions of the human brain. The letter “Z” stands for zero and refers to electrodes placed along the midline. Even numbers (e.g. 2, 4, 6 and 8) refer to electrode positions on the right cerebral hemisphere, whereas odd numbers (e.g. 1, 3, 5 and 7) refer to those on the left cerebral hemisphere.

Optional user interface 50 is in one embodiment arranged to graphically present information on a display (not shown). Optional user interface 50 is arranged to provide the user a graphical interface for setting up of the modes/parameters and various operational criteria of system 10.

Optional patient stimulation module 70 is in communication with analyzing module 60. In one embodiment, optional patient stimulation module 70 comprises an auditory stimulation device arranged to produce an audible sound wave which is optionally output by a speaker or ear phone (not shown), without limitation. In another embodiment, optional patient stimulation module 70 comprises a somatosensory stimulation device arranged to stimulate the somatosensory system.

FIG. 1B illustrates a high level block diagram of analyzing module 60, according to certain embodiments. Analyzing module 60 comprises: an optional pre-processing functionality 80; an EEG activity detection functionality 90; a memory 95; an optional comparing functionality 100; a difference determining functionality 110; and a neurological deterioration determination functionality 120. Optional pre-processing functionality 80 is arranged to process EEG signals received from electrodes 30 and 40. In one embodiment, optional pre-processing functionality 80 comprises: an amplifier arranged to amplify the received EEG signals; an A/D converter arranged to digitize the amplified EEG signals; and a filter 85 arranged to extract different frequency bands from the digitized EEG signals. It is to be noted that the above order of amplifying, digitizing and filtering is not limiting and the pre-processing can be performed in any order without exceeding the scope.

In one non-limiting embodiment, filter 85 is arranged to divide EEG signals received from electrodes 30, 40 into four frequency bands by splitting the received EEG into 4 portions and then providing the appropriate band pass filter. In one non-limiting embodiment the four frequency bands are Delta, Theta, Alpha and Beta, as described above.

In another embodiment, filter 85 is arranged to divide the received EEG signals into a greater number of frequency bands. In one further embodiment, the EEG signals are divided into sub-bands of the above four frequency bands. Advantageously, such a sub-division provides for further resolution of the activity within each standard band. In another further embodiment, the EEG signals are filtered into frequency bands exhibiting generally equal widths, across a spectrum of 0.1 Hz-40 Hz. Advantageously, such a sub-division provides for a systematic overview of the EEG activity, which is beneficial upon accumulation of significant amount of clinical data.

The processed information is analyzed by analyzing module 60, as will be described further below. In one embodiment, optional pre-processing functionality 80 is further arranged to detect one or more of: failure or malfunction of any of electrodes 30, 40; a low level of energy in the energy sources of electrodes 30, 40; and incorrect placement of electrodes 30, 40 on the head of patient 20.

In one embodiment, neurological deterioration determination functionality 120 comprises a communication module 130 arranged to communicate with an external medical system. In one embodiment, communication module 130 is arranged to communicate with the external medical system via short message service (SMS) or phone call. In one embodiment, communication module 130 comprises any of: a cellular network; a frequency antenna; a subscriber identification module (SIM); an infrared transmitters; a wireless personal area network (WPAN) transmitter; a wireless local area network (WLAN) transmitter; a wide area network (WAN) transmitter; a computer network connection; a cable modem and/or LAN socket interface for establishing a cable connection and/or a telephony connection, without limitation. In one embodiment, neurological deterioration determination functionality 120 is arranged to graphically present information on a display portion of optional user interface 50 (not shown).

In operation, as will be described below in relation to FIGS. 1C-1E, analyzing module 60 is arranged to determine a neurological deterioration responsive to any of a plurality of analyzing methods of received EEG samples from electrodes 30 and 40. In particular, the neurological deterioration is determined by detecting, without limitation, one of: a difference between a characteristic of the received EEG samples and a baseline value, the characteristics being, without limitation, any of the amplitude of the EEG samples, the difference in the amplitudes of EEG samples of different electrodes, the variability of the amplitudes of the EEG samples, the rate of change of the amplitudes of the EEG samples and the correlation between EEG samples of different electrodes of between differences of EEG samples of different electrodes; a change in any of the above characteristics, without limitation, over a predetermined period of time; and a difference in a characteristic of different electrodes positioned in different cerebral locations, such as in opposing cerebral hemispheres, without limitation and as will be described further hereinto below.

As known in the art of EEG analysis, EEG signals are analyzed in reference to other EEG signals. In one non-limiting embodiment, each EEG signal is analyzed with reference to a reference electrode, such as electrodes positioned in the A1 and/or A2 positions. In another non-limiting embodiment, a plurality of electrodes are positioned on the head of patient 20 and each EEG signal is analyzed in relation to an average of the EEG signals received from the plurality of electrodes. In another non-limiting embodiment, as will be described below in relation to FIG. 1D, the EEG signal of each electrode 30 is analyzed in relation to another electrode 30 and the EEG signal of each electrode 40 is analyzed in relation to another electrode 40.

FIG. 1C illustrates a first embodiment of the operation of system 10. In stage 1000, electrodes 30 are disposed along the left side of the head of patient 20 and electrodes 40 are disposed along the right side of the head of patient 20. In one non-limiting embodiment, electrodes 30 are positioned in the F3, C3, T3, P3 and O1 positions and electrodes 40 are positioned in the F4, C4, T4, P4 and O2 positions. Each electrode 30 is associated with the particular electrode 40 positioned in the same region, i.e. an electrode 30 is positioned at F3 and an electrode 40 is positioned at F4; an electrode 30 is positioned at C3 and an electrode 40 is positioned at C4; an electrode 30 is positioned at P3 and an electrode 40 is positioned at P4; and an electrode 30 is positioned at O1 and an electrode 40 is positioned at O2. Preferably, electrodes 30 and 40 are positioned such that patient 20 is able to sleep during the operation of system 10.

In stage 1010, EEG samples are received from electrodes 30 and electrodes 40 at analyzing module 60 at a plurality of times. In one embodiment, the received EEG samples are pre-processed by optional pre-processing functionality 80, as described above and a plurality of frequency bands are extracted from the received EEG signals. In one embodiment, the received EEG samples are stored on memory 95, optionally after being pre-processed. In one embodiment, optional pre-processing functionality 80 is further arranged to compare the amplitude of the received EEG samples with a predetermined minimum value, the predetermined minimum value being predetermined as the value wherein an EEG signal exhibiting such an amplitude is too weak to provide useful information. In the event the received EEG samples of one or more electrodes 30, 40 exhibit an amplitude less than the predetermined minimum value for at least a predetermined time period, optional patient stimulation module 70 is arranged to provide stimulation to patient 20, as will be described below in relation to FIG. 1E, and stage 1010 is again performed. When patient 20 is in a state of reduced arousal, EEG activity may be very low, i.e. the amplitude is below the predetermined minimum value. Therefore, stimulation of patient 20 will cause patient 20 to transcend to a state of increased arousal, where EEG activity will increase so as to exceed the predetermined minimum value.

In one embodiment, optional pre-processing functionality 80 is further arranged to calculate the standard deviation of the received EEG samples. In the event that the calculated standard deviation is greater than a predetermined value, the particular electrode 30, 40 is determined to be outputting noise and optional pre-processing functionality 80 is arranged to output a noise detection signal for the particular electrode 30 or 40. In one embodiment, the output noise detection signal is displayed on optional user interface 50. In another alternate embodiment, the output noise detection signal is transmitted to the particular electrode, which as described above optionally comprises an indicator, and the indicator is arranged to indicate the particular electrode 30, 40 is producing noise. Thus, the operator can adjust the position of the particular electrode 30, 40 in response to the output noise detection signal. In one embodiment, if noise is detected for less than a predetermined time period, a noise detection signal is not output, however the received EEG samples are discarded.

As described above, in one non-limiting embodiment each EEG signal is referenced to a reference electrode such as an electrode positioned in the A1 or A2 position. In another non-limiting embodiment, each EEG signal is referenced to an average of the EEG signals received from a plurality electrodes positioned around the head of patient 20.

In optional stage 1015, optional pre-processing functionality 80 is further arranged to determine the current sleep stage of patient 20, responsive to the received EEG samples from electrodes 30 and 40. As described above, each sleep stage exhibits particular characteristics of EEG activity, optional pre-processing functionality 80 arranged to determine the current sleep stage according to the above defined characteristics. As will be described below in relation to stage 1030, a baseline value is utilized for analyzing the received EEG samples. In one embodiment, memory 95 contains a plurality of baseline values, each associated with a particular sleep stage. A particular baseline value is selected responsive to the determined current sleep stage of patient 20 and according to appropriate parameters, as will be described below.

In stage 1020, optional comparing functionality 100 determines the correlation between the EEG samples of each electrode 30 and the EEG samples of the associated electrode 40. In one non-limiting embodiment, the correlation is determined for the delta frequency band. Optionally, the correlation is determined for each extracted frequency band of the EEG samples. In one non-limiting embodiment, the correlation is determined over about 1500 received EEG samples of stage 1010. In one non-limiting embodiment, the correlation is determined over a period greater than a second. In another non-limiting embodiment, the correlation is determined over a period of less than 50 milliseconds. In one embodiment, the at least one determined correlation is stored on memory 95. Advantageously, the impact of noise on the determined correlation is minimal.

In stage 1030, difference determining functionality 110 is arranged to determine the difference between the correlation of stage 1020 and a baseline value. As described above, the determining is performed for each electrode 30 and the associated electrode 40. As described above, in one embodiment, the baseline value is further determined responsive to the determined sleep stage of patient 20. In the embodiment where the correlation was determined for each of a plurality of frequency bands, the difference is determined for each frequency band. In one embodiment, the baseline value is a general population baseline. In particular, values of the correlation between EEG signals received from an electrode in communication with the left cerebral hemisphere and a corresponding electrode in communication with the right cerebral hemisphere of a plurality of patients is stored on memory 95. The difference between the correlation of stage 1020 and the stored correlation values is determined. In one embodiment, the difference is determined by comparing the correlation of stage 1020 with an average of the stored correlation values.

In another embodiment, the baseline value is a baseline of patient 20. In particular, values of the correlation between EEG signals received from an electrode 30 and an electrode 40 of patient 20 is stored on memory 95. In one embodiment, stage 1010 as described above is continuously performed and the stored correlation values are from previous correlation determinations of stage 1020. In another embodiment, the stored correlation values comprise historical correlation values of EEG samples of patient 20 in previous monitoring sessions. The difference between the correlation of stage 1020 and the stored correlation values is determined. In one embodiment, the difference is determined by comparing the correlation of stage 1020 with an average of the stored correlation values.

In stage 1040, neurological deterioration determination functionality 120 is arranged to determine if a neurological deterioration is present in patient 20. In particular, neurological deterioration determination functionality 120 is arranged to compare the determined difference of stage 1030 to a predetermined value. As described above, the comparison is performed for the received EEG samples of each electrode 30 and associated electrode 40. In the embodiment where a difference was determined for each of a plurality of frequency bands, each frequency band difference is compared to the predetermined value and in one embodiment is compared to a particular one of a plurality of predetermined values, each associated with a respective frequency band.

In the event the determined difference of stage 1030 is greater than the predetermined value, i.e. a neurological deterioration is detected, neurological deterioration determination functionality 120 is arranged to output a neurological deterioration signal. In one embodiment, the neurological deterioration signal is output when the difference of stage 1030 determined for the received EEG samples of at least one electrode 30 and the received EEG samples of the associated electrode 40 is greater than the predetermined value. In another embodiment, the neurological deterioration signal is output when the difference of stage 1030 determined for the received EEG samples of a plurality of electrodes 30 and the received EEG samples of the associated electrodes 40 are greater than the predetermined value. In the embodiment where the comparison was performed for each of a plurality of frequency bands, optionally the neurological deterioration signal is output when at least one of the determined differences of stage 1030 is greater than the predetermined value. In another embodiment, the neurological deterioration signal is output when a plurality of the determined differences of stage 1030 is greater than the predetermined value. In the embodiment where, as described above, neurological deterioration determination functionality 120 comprises communication module 130, the neurological deterioration signal is communicated to an external medical system.

As described above, several characteristic changes in the EEG activity of a patient following neurological deteriorations are known to the prior art. Additionally, characteristic changes in the EEG activity of an individual during the different stages of sleep are known. As described above and below, in one embodiment the baseline value is selected responsive to the determined current sleep stage of patient 20, the baseline value and any associated predetermined value or time period being determined according to the relation between the characteristic changes of the EEG signals during sleep, optionally according to the particular sleep stage, and the characteristic changes of the EEG signals of patient 20 suffering from a neurological deterioration, such as CVA. However, the inventors have discovered that detection of the known characteristic changes while the patient is asleep is challenging because of the relative similarity between characteristic changes of the EEG signals during sleep and characteristic changes of the EEG signals after a neurological deterioration. The above and below methods of determining the correlation between the EEG signals, or functions of EEG signals, received from different electrodes successfully overcome these challenges.

As will be described below in relation to FIG. 1F, in one embodiment optional patient stimulation module 70 is arranged to stimulate patient 20 responsive to the output neurological deterioration signal and stage 1010 as described above is again performed. In the event that in stage 1040 the determined difference of stage 1030 is not greater than the predetermined value, stage 1010 as described above is performed.

Although the above has been described in an embodiment of comparing EEG samples received from an electrode 30 with EEG samples received from an electrode 40, this is not meant to be limiting in any way and in another embodiment the correlation between EEG samples of a plurality of electrodes 30 or a plurality of electrodes 40 is determined, without exceeding the scope.

Thus, the method of FIG. 1C provides an arrangement of analyzing module 60 to determine a neurological deterioration responsive to a determination of the correlation between EEG samples of different electrodes and responsive to one of: a difference between the correlation and a baseline value; and a change in the determined correlation over a predetermined period of time.

FIG. 1D illustrates a second embodiment of the operation of system 10. In stage 2000, at least one pair of electrodes 30 are disposed along the left side of the head of patient 20 and at least one pair of electrodes 40 are disposed along the right side of the head of patient 20. In one non-limiting embodiment, electrodes 30 are positioned in the F3 and C3 positions and corresponding electrodes 40 are positioned in the F4 and C4 positions. As described above in relation to FIG. 1C, each pair of electrodes 30 and an associated pair of electrodes 40 are positioned in the corresponding region. Preferably, electrodes 30 and 40 are positioned such that patient 20 is able to sleep during the operation of system 10.

In stage 2010, EEG samples are received from electrodes 30 and electrodes 40 at analyzing module 60 at a plurality of times. In one embodiment, the received EEG samples are pre-processed by optional pre-processing functionality 80, as described above and a plurality of frequency bands are extracted from the received EEG signals. In one embodiment, the received EEG samples are stored on memory 95, optionally after being pre-processed. As described above in relation to stage 1010, in one embodiment, optional pre-processing functionality 80 is further arranged to compare the amplitude of the received EEG samples with a predetermined minimum value, the predetermined minimum value being the value wherein an EEG signal exhibiting such an amplitude is too weak to provide useful information. In the event the received EEG samples of one or more electrodes 30, 40 exhibit an amplitude less than the predetermined minimum value for at least a predetermined time period, optional patient stimulation module 70 is arranged to provide stimulation to patient 20, as will be described below in relation to FIG. 1F, and stage 2010 is again performed. As described above, optional pre-processing functionality 80 is arranged to output a noise detection signal if optional pre-processing functionality 80 determines that a particular electrode 30, 40 is outputting noise. In one embodiment, as described above in relation to optional stage 1015, the current sleep stage of patient 20 is determined.

In stage 2020, optional comparing functionality 100 is arranged to compare the received EEG samples from a first of each pair of electrodes 30 to the received EEG samples from the second of the respective pair of electrodes 30. In one non-limiting embodiment, the comparison is performed in the alpha frequency band. In the embodiment where a plurality of frequency bands were extracted from each received EEG signal, the comparison is optionally performed for each frequency band.

In one embodiment, the comparison comprises determining the difference between the amplitudes of the received EEG samples from the first of the pair of electrodes 30 with the amplitudes of the received EEG samples from the second of the pair of electrodes 30. In one embodiment, the difference between the amplitudes of each sample are determined and in one further embodiment an average of a plurality of determined differences are determined. In another embodiment, prior to the comparison, optional comparing functionality 100 is arranged to calculate an average of the amplitude values of a plurality of the received EEG samples from each electrode 30 and determine the difference between the average of the amplitudes of the received EEG samples of the first of the pair of electrodes 30 and the average of the amplitudes of the received EEG samples of the second of the pair of electrodes 30. In one embodiment, the determined difference is stored on memory 95.

In another embodiment, the comparison comprises determining the correlation between the received EEG samples from the first electrode of the pair of electrodes 30 and the received EEG samples from the second electrode of the pair of electrodes 30. In one embodiment, the determined correlation is stored on memory 95. As described above in relation to stage 1020, one advantage of determining correlation is that the impact of noise on the determined correlation is minimal.

In stage 2030, optional comparing functionality 100 is arranged to compare the received EEG samples from a first of each pair of electrodes 40 to the received EEG samples from the second of the respective pair of electrodes 40. In the embodiment where a plurality of frequency bands were extracted from each received EEG signal, the comparison is performed for each frequency band.

In one embodiment, the comparison comprises determining the difference between the amplitudes of the received EEG samples from the first electrode of the pair of electrodes 40 and the amplitudes of the received EEG samples from the second electrode of the pair of electrodes 40. In one embodiment, the difference between the amplitudes of each sample are determined and in one further embodiment an average of the determined differences are calculated over a predetermined time period. In another embodiment, prior to the comparison, optional comparing functionality 100 is arranged to calculate an average of the received EEG samples from each electrode 40 over a predetermined time period and determine the difference between the average of the amplitudes of the received EEG samples of the first electrode of the pair of electrodes 40 and the average of the amplitudes of the received EEG samples of the second electrode of the pair of electrodes 40. In one embodiment, the determined difference is stored on memory 95.

In another embodiment, the comparison comprises determining the correlation between the received EEG samples from the first electrode of the pair of electrodes 40 and the received EEG samples from the second electrode of the pair of electrodes 40. In one embodiment, the determined correlation is stored on memory 95.

In stage 2040, difference determining functionality 110 is arranged to determine the difference between the comparison of stage 2020 and the comparison of stage 2030 for each pair of electrodes 30 and associated pair of electrodes 40, the difference being referred hereinto below as the comparison difference. In one embodiment, the comparison difference is determined as an absolute difference and in another embodiment the comparison difference is determined as a ratio. In one embodiment, the comparison difference is determined as a function of the determined difference, such as an average or a standard deviation, without limitation. In another embodiment, the comparison difference is determined as the correlation between the comparison of stage 2030 and the comparison of stage 2020.

Difference determining functionality 110 is further arranged to determine the difference between the determined comparison difference and a baseline value. In the embodiment where the comparison of stages 2020 and 2030 was performed for each of a plurality of frequency bands, the comparison difference and the difference between the comparison difference and the baseline value is determined for each frequency band. In the embodiment where in stages 2020-2030 the amplitudes of received EEG signals from pairs of electrodes are compared, the baseline value comprises a value representing a difference between comparisons of EEG signals from pairs of electrodes. In the embodiment where in stages 2020-2030 the correlation of received EEG signals from pairs of electrodes are compared, the baseline value comprises a value representing a difference between correlations of EEG signals from pairs of electrodes.

In one embodiment, as described above in relation to stage 1030, the baseline value is a general population baseline. In another embodiment, as described above, the baseline value is a baseline of patient 20. As described above, in one embodiment the baseline value is selected from a plurality of baseline values responsive to a determination of the current sleep stage of patient 20.

In stage 2050, neurological deterioration determination functionality 120 is arranged to determine if a neurological deterioration is present in patient 20. In particular, neurological deterioration determination functionality 120 is arranged to compare the determined difference of stage 2040 to a predetermined value. The comparison is performed for the received EEG samples of each pair of electrodes 30 and the associated pair of electrodes 40. In the embodiment where in stage 2040 a difference was determined for each of a plurality of frequency bands, each difference is compared to a predetermined value and in one embodiment is compared to a particular one of a plurality of predetermined values, each associated with a respective frequency band.

In the event the determined difference of stage 2040 is greater than the predetermined value, i.e. a neurological deterioration is detected, neurological deterioration determination functionality 120 is arranged to output a neurological deterioration signal. In one embodiment, the neurological deterioration signal is output when the difference of stage 2040 determined for the received EEG samples of at least one pair of electrodes 30 and the received EEG samples of the associated pair of electrodes 40 is greater than the predetermined value. In another embodiment, the neurological deterioration signal is output when the difference of stage 2040 determined for the received EEG samples of a plurality of pairs electrodes 30 and the received EEG samples of the associated pairs electrodes 40 are greater than the predetermined value. In the embodiment where the comparison was performed for each of a plurality of frequency bands, optionally the neurological deterioration signal is output when at least one of the determined differences of stage 2040 is greater than the predetermined value. In another embodiment, the neurological deterioration signal is output when a plurality of the determined differences of stage 2040 is greater than the predetermined value. In the embodiment where, as described above, neurological deterioration determination functionality 120 comprises communication module 130, the neurological deterioration signal is communicated to an external medical system.

In another alternative embodiment, the neurological deterioration is determined by comparing the outcomes of any of stages 2020 and 2030 with a baseline value. In particular, the determined amplitude difference or determined correlation is compared with a baseline value and the neurological deterioration signal is output responsive to the comparison, as described below in relation to FIG. 1E. In another alternative embodiment, as described below, the neurological deterioration signal is output responsive to a change in the determined amplitude difference or determined correlation lasting for more than a predetermined time period.

As will be described below in relation to FIG. 1F, in one embodiment optional patient stimulation module 70 is arranged to stimulate patient 20 responsive to the output neurological deterioration signal, thereby increasing the EEG activity of patient 20, and stage 2010 as described above is performed. In the event that in stage 1040 the determined difference of stage 1030 is not greater than the predetermined value, stage 2010 as described above is performed.

FIG. 1E illustrates a third embodiment of the operation of system 10. In stage 3000, at least one electrode 30 is disposed on the head of patient 20, as described above in relation to FIGS. 1C-1D. In one embodiment, a plurality of electrodes 30 are disposed on the head of patient 20. In one embodiment, as described above, electrodes 30 are positioned in accordance with the international 10-20 system.

In stage 3010, EEG samples are received from electrodes 30 at analyzing module 60, preferably at a plurality of times. In one embodiment, the received EEG samples are pre-processed by optional pre-processing functionality 80, as described above and a plurality of frequency bands are extracted from the received EEG signals. In one embodiment, the received EEG samples are stored on memory 95, optionally after being pre-processed. As described above in relation to stage 1010, in one embodiment, optional pre-processing functionality 80 is further arranged to compare the amplitude of the received EEG samples with a predetermined minimum value, the predetermined minimum value being the value wherein an EEG signal exhibiting such an amplitude is too weak to provide useful information. In the event the received EEG samples of one or more electrodes 30 exhibit an amplitude less than the predetermined minimum value for at least a predetermined time period, optional patient stimulation module 70 is arranged to provide stimulation to patient 20, as will be described below in relation to FIG. 1F, and stage 3010 is again performed. As described above, optional pre-processing functionality 80 is arranged to output a noise detection signal if optional pre-processing functionality 80 determines that a particular electrode 30, 40 is outputting noise. As described above, in one embodiment the current sleep stage of patient 20 is determined.

As described above, in one non-limiting embodiment each EEG signal is referenced to a reference electrode such as an electrode positioned in the A1 or A2 position. In another non-limiting embodiment, each EEG signal is referenced to an average of the EEG signals received from a plurality electrodes positioned around the head of patient 20.

In stage 3020, the amplitudes of the received EEG signals of each electrode 30 are compared to a baseline value. In one non-limiting embodiment, the comparison is performed in the alpha frequency band. In the embodiment where a plurality of frequency bands were extracted from each received EEG signal, the comparison is optionally performed for each frequency band. As described above, in one embodiment the baseline value is selected from among a plurality of baseline values responsive to a determination of the current sleep stage of patient 20.

In one embodiment, difference determining functionality 110 is arranged to determine the difference between an average of the amplitude values of the received EEG signals from the electrode 30, over a predetermined time period, and the baseline value, the baseline value being a baseline value of amplitude values of EEG signals. In one embodiment, as described above in relation to stage 1030, the baseline value is a general population baseline. In another embodiment, as described above, the baseline value is a baseline of patient 20.

In another embodiment, optional comparing functionality 100 is arranged to compare the amplitude of each received EEG sample from the electrode 30 to the amplitude of the previous EEG sample, preferably stored on memory 95. Optional comparing functionality 100 is further arranged to determine the rate of change of the amplitude of the EEG signal, responsive to successive comparisons of amplitudes of samples of the EEG signal over a predetermined time period. Difference determining functionality 110 is arranged to determine the difference between the determined rate of change and the baseline value, the baseline value being a baseline value of rates of change of amplitude values of EEG signals. In one embodiment, as described above in relation to stage 1030, the baseline value is a general population baseline. In another embodiment, as described above, the baseline value is a baseline of patient 20.

In another embodiment, optional comparing functionality 100 is arranged to determine a plurality of averages of the amplitudes of each received EEG signal, each average determined over a predetermined time period. Optional comparing functionality 100 is further arranged to determine the rate of change of the values of the determined averages. Difference determining functionality 110 is arranged to determine the difference between the determined rate of change and the baseline value, the baseline value being a baseline value of rates of change of averages of amplitude values of EEG signals. In one embodiment, as described above in relation to stage 1030, the baseline value is a general population baseline. In another embodiment, as described above, the baseline value is a baseline of patient 20.

In another embodiment, optional comparing functionality 100 is arranged to compare the amplitude of each received EEG sample from the electrode 30 to the amplitude of the previous EEG sample, preferably stored on memory 95. Optional comparing functionality 100 is further arranged to determine the variability of the amplitude of the EEG signal, responsive to successive comparisons of amplitudes of samples of the EEG signal over a predetermined time period. Difference determining functionality 110 is arranged to determine the difference between the determined variability and the baseline value, the baseline value being a baseline value of the variability of amplitude values of EEG signals. In one embodiment, as described above in relation to stage 1030, the baseline value is a general population baseline. In another embodiment, as described above, the baseline value is a baseline of patient 20.

In stage 3030, neurological deterioration determination functionality 120 is arranged to determine if a neurological deterioration is present in patient 20. In particular, neurological deterioration determination functionality 120 is arranged to compare the determined difference of stage 3020 to a predetermined value. The comparison is performed in relation to the received EEG samples of each electrode 30. In the embodiment where in stage 3020 a difference was determined for each of a plurality of frequency bands, each difference is compared to a predetermined value and in one embodiment is compared to a particular one of a plurality of predetermined values, associated with the particular frequency band.

In the event the determined difference of stage 3020 is greater than the predetermined value, i.e. a neurological deterioration is detected, neurological deterioration determination functionality 120 is arranged to output a neurological deterioration signal. In one embodiment, the neurological deterioration signal is output when the difference of stage 3020 determined for the received EEG samples of at least one electrode 30 is greater than the predetermined value. In another embodiment, the neurological deterioration signal is output when the differences of stage 3020 determined for the received EEG samples of a plurality of electrodes 30 are greater than the predetermined value. In the embodiment where the comparison was performed for each of a plurality of frequency bands, optionally the neurological deterioration signal is output when at least one of the determined differences of stage 3020 is greater than the predetermined value. In another embodiment, the neurological deterioration signal is output when a plurality of the determined differences of stage 3020 is greater than the predetermined value. In one embodiment, neurological deterioration determination functionality 120 is arranged to output the neurological deterioration signal responsive to the determined difference of stage 3020 being greater than the predetermined value for at least a predetermined time period. In the embodiment where, as described above, neurological deterioration determination functionality 120 comprises communication module 130, the neurological deterioration signal is communicated to an external medical system.

In one alternative embodiment, the above described method of FIG. 1E is performed as described above in relation to FIGS. 1C and 1D. In particular, any of the determined amplitudes, rates of change and variabilities of stage 3020 for a particular electrode are compared to the respective determined values of another electrode. In one embodiment, the comparison is performed between electrodes receiving EEG signals from opposing cerebral hemispheres, as described above in relation to electrodes 30 and 40 of FIGS. 1C and 1D.

As will be described below in relation to FIG. 1F, in one embodiment optional patient stimulation module 70 is arranged to stimulate patient 20 responsive to the output neurological deterioration signal, thereby increasing the EEG activity of patient 20, and stage 3010 as described above is performed. In the event that in stage 3030 the determined difference of stage 3020 is not greater than the predetermined value, stage 3010 as described above is performed.

FIG. 1F illustrates a high level flow chart of a fourth embodiment of the method of operation of system 10. In stage 4000, as described above, optional patient stimulation module 70 is provided. As described above, optional patient stimulation module 70 is in communication with analyzing module 60 and is arranged to receive therefrom a plurality of signals. In particular, optional patient stimulation module 70 is arranged to receive from analyzing module 60 a non-stimulated state patient stimulation signal. Responsive to the received non-stimulated state patient stimulation signal, optional patient stimulation module 70 is arranged to provide non-awakening stimulation to patient 20. As described above, in one embodiment an auditory stimulation is provided to patient 20 and in another embodiment a somatosensory stimulation is provided to patient 20. The provided stimulation is arranged to at least partially increase the EEG activity of patient 20, while not awakening patient 20.

In stage 4010, system 10 is in a non-stimulated mode. In one embodiment, electrodes 30 and 40 are attached to the head of patient 20, patient 20 goes to sleep and system 10 is arranged to be in the non-stimulated mode. In one embodiment, the mode of system 10 can be adjusted responsive to a user input at optional user interface 50. EEG samples are received from electrodes attached to the head of patient 20, as described above in any of stages 1010, 2010 and 3010. In stage 4020, analyzing module 60 is arranged to determine if a neurological deterioration is present in patient 20, as described above in relation to stages 1020-1050, 2020-2060 and 3020-3040. In the event a neurological deterioration isn't determined to be present, stage 4010 is again performed.

In the event a neurological deterioration is determined, i.e. a neurological deterioration signal is output as described above in relation to stages 1050, 2060, and 3040, in stage 4030 a non-stimulated state patient stimulation signal is provided to optional patient stimulation module 70 and optional patient stimulation module 70 is arranged to stimulate patient 20 responsive thereto. As described above in relation to stage 1010, stimulation causes an increase in EEG activity of patient 20. System 10 is now in a stimulated mode.

In stage 4040, EEG samples are received, as described above in relation to stage 4010 and in stage 4050 analyzing module 60 is arranged to determine if a neurological deterioration is present in patient 20, as described above in relation to stage 4010. The increased EEG activity caused by the stimulation of patient 20 allows for determination of neurological deterioration with greater accuracy. Preferably, the baseline values are adjusted for the stimulated state of patient 20. In the event a neurological deterioration isn't determined to be present in patient 20, stage 4040 is again performed. In the event a neurological deterioration is determined to be present in patient 20, i.e. a neurological deterioration signal is output as described above in relation to stages 1050, 2060, and 3040, in stage 4060 a stimulated state patient stimulation signal is provided to optional patient stimulation module 70 and optional patient stimulation module 70 is arranged to provide awakening stimulation to patient 20 responsive thereto. As described above, the provided awakening stimulation is such that patient 20 is fully aroused. System 10 is now in an awakened mode.

In stage 4070, EEG samples are received, as described above in relation to stage 4010 and in stage 4080 analyzing module 60 is arranged to determine if a neurological deterioration is present in patient 20, as described above in relation to stage 4020. Preferably, the baseline values are adjusted for the awakened state of patient 20. The increased EEG activity caused by the awakening of patient 20 allows for determination of neurological deterioration with greater accuracy. In the event a neurological deterioration isn't determined to be present in patient 20, stage 4070 is again performed. In the event a neurological deterioration is determined to be present in patient 20, in stage 4090 neurological deterioration determination functionality 120 of analyzing module 60 is arranged to output a neurological deterioration signal, as described above in relation to stages 1050, 2060 and 3040, preferably in cooperation with communication module 130 thereby alerting medical assistance. As described, in one embodiment communication module 130 comprises any, or all, of: a connection to an audible output device, such as a speaker; a connection to a graphical output device, such as a display; a connection to short messaging service; a connection to a telephony network; a connection to the Internet; and a connection to a medical network, such as a computer network of a medical facility.

### EXAMPLES

Values of exemplary parameters Y1 and C1, which are further defined below, of fronto-temporal activity in the right hemisphere versus the left hemisphere, from a patient exhibiting a CVA in the left cerebral hemisphere obtained over 8 minutes of sleep were analyzed by the above methods described in FIG. 1D.

### Example 1

Parameter Y1 was defined as the ratio of the difference (Diff) between the differences between the amplitude of the EEG signals received from electrodes positioned on a patient in the F7 and T3 positions (SIGNAL Left) and the amplitude of the EEG signals received from electrodes positioned on the patient in the F8 and T4 positions (SIGNAL Right), over 8 consecutive timeframes of 1 min. A threshold, i.e. the baseline value, was defined as at least 20% exceedance, successively over 8 timeframes of 1 min, in the amplitude of the EEG signals in one hemisphere compared to the counterpart hemisphere.

### Example 2

Parameter C1 was defined as the standard deviation (STDV) of 8 values of parameter Y1 over a period of 1 minute. A threshold. i.e. the baseline value, for parameter C1 was defined as the numerical value of 13. The raw EEG data and mathematical calculations are presented in Table 1 for the CVA patient and in Table 2 for the healthy individual.

TABLE 1

| | SIGNAL [μv] | | CALCULATION | | EVALUATION | | |
|---|---|---|---|---|---|---|---|
| # | Left | Right | Diff | Ratio % | % > 20 | % < −20 | STDV |
| 1 | 15395.10 | 34435.56 | 19040.46 | 55.29 | YES | | 12.4731 |
| 2 | 54414.73 | 134655.48 | 80240.75 | 59.59 | YES | | |
| 3 | 20772.39 | 51131.28 | 30358.90 | 59.37 | YES | | |
| 4 | 26195.44 | 48888.81 | 22693.38 | 46.42 | YES | | |
| 5 | 21531.42 | 31931.31 | 10399.89 | 32.57 | YES | | |
| 6 | 30253.13 | 85586.11 | 55332.99 | 64.65 | YES | | |
| 7 | 56747.82 | 83208.22 | 26460.40 | 31.80 | YES | | |
| 8 | 25592.36 | 46821.94 | 21229.58 | 45.34 | YES | | |

TABLE 2

| | SIGNAL [μv] | | CALCULATION | | EVALUATION | | |
|---|---|---|---|---|---|---|---|
| # | Left | Right | Diff | Ratio % | % > 20 | % < −20 | STDV |
| 1 | 348499.41 | 41039.41 | −307460.01 | −749.18 | | YES | |
| 2 | 19166.70 | 13237.75 | −5928.95 | −44.79 | | YES | |
| 3 | 37968.42 | 26550.48 | −11417.95 | −43.00 | | YES | |
| 4 | 17522.29 | 17138.37 | −383.91 | −2.24 | | | |
| 5 | 13929.91 | 10998.76 | −2931.15 | −26.65 | | YES | |
| 6 | 13561.70 | 10549.64 | −3012.06 | −28.55 | | YES | |
| 7 | 21069.82 | 13129.83 | −7939.99 | −60.47 | | YES | |
| 8 | 14138.61 | 14797.00 | 658.39 | 4.45 | | | 255.6 |
| 9 | 200326.16 | 85161.14 | −115165.02 | −135.23 | | YES | 43.42 |
| 10 | 16631.65 | 15596.18 | −1035.47 | −6.64 | | | 45.14 |
| 11 | 9986.83 | 13229.73 | 3242.90 | 24.51 | YES | | 49.97 |
| 12 | 11098.42 | 13436.00 | 2337.58 | 17.40 | | | 51.91 |
| 13 | 6854.94 | 6110.98 | −743.96 | −12.17 | | | 52.15 |
| 14 | 8352.09 | 10211.83 | 1859.74 | 18.21 | | | 54.22 |
| 15 | 8660.54 | 11542.57 | 2882.03 | 24.97 | YES | | 53.24 |
| 16 | 55423.76 | 20587.51 | −34836.25 | −169.21 | | YES | 77.35 |
| 17 | 131965.82 | 25816.51 | −106149.31 | −411.17 | | YES | 154.3 |
| 18 | 20857.44 | 22429.52 | 1572.08 | 7.01 | | | 155.1 |
| 19 | 20642.29 | 32493.92 | 11851.64 | 36.47 | YES | | 156.1 |
| 20 | 11894.26 | 8574.04 | −3320.21 | −38.72 | | YES | 153.3 |
| 21 | 4281.03 | 9350.69 | 5069.66 | 54.22 | YES | | 158.5 |
| 22 | 8852.08 | 6113.17 | −2738.91 | −44.80 | | YES | 155.6 |
| 23 | 6173.44 | 7701.66 | 1528.21 | 19.84 | | | 155.2 |
| 24 | 5947.70 | 7418.27 | 1470.57 | 19.82 | | | 152 |
| 25 | 4335.55 | 5129.23 | 793.68 | 15.47 | | | 34.33 |
| 26 | 84831.44 | 536286.79 | 451455.35 | 84.18 | YES | | 43.44 |
| 27 | 136595.61 | 87420.04 | −49175.57 | −56.25 | | YES | 49.8 |
| 28 | 4011.96 | 4866.96 | 855.00 | 17.57 | | | 46.32 |
| 29 | 372805.82 | 118087.08 | −254718.74 | −215.70 | | YES | 90.18 |
| 30 | 25136.51 | 11713.36 | −13423.14 | −114.60 | | YES | 96.1 |
| 31 | 6261.59 | 6328.76 | 67.18 | 1.06 | | | 94.97 |
| 32 | 720246.18 | 62076.46 | −658169.72 | −1060.26 | | YES | 373 |
| 33 | 11517.54 | 10624.40 | −893.15 | −8.41 | | | 371.4 |
| 34 | 8840.02 | 7427.08 | −1412.94 | −19.02 | | | 363.1 |
| 35 | 5410.06 | 5604.57 | 194.51 | 3.47 | | | 366.6 |
| 36 | 6614.26 | 9843.16 | 3228.90 | 32.80 | YES | | 367.8 |
| 37 | 6396.83 | 9248.38 | 2851.55 | 30.83 | YES | | 374 |
| 38 | 10608.41 | 12292.20 | 1683.79 | 13.70 | | | 378 |
| 39 | 20224.14 | 25951.84 | 5727.69 | 22.07 | YES | | 379.1 |
| 40 | 77219.47 | 102740.01 | 25520.54 | 24.84 | YES | | 18.92 |
| 41 | 315763.79 | 53636.98 | −262126.81 | −488.71 | | YES | 179.1 |
| 42 | 11288.84 | 13074.92 | 1786.08 | 13.66 | | | 180.2 |
| 43 | 19905.79 | 19978.27 | 72.47 | 0.36 | | | 180.1 |
| 44 | 9396.86 | 8175.03 | −1221.83 | −14.95 | | | 178 |
| 45 | 7774.52 | 7001.79 | −772.73 | −11.04 | | | 175.8 |
| 46 | 7021.28 | 56320.95 | 49299.67 | 87.53 | YES | | 181.8 |
| 47 | 10285.06 | 16014.77 | 5729.71 | 35.78 | YES | | 182.6 |
| 48 | 26885.44 | 7668.03 | −19217.40 | −250.62 | | YES | 193.4 |
| 49 | 10351.46 | 5453.85 | −4897.62 | −89.80 | | YES | 102.7 |
| 50 | 11371.17 | 10783.52 | −587.65 | −5.45 | | | 101.8 |
| 51 | 8739.90 | 5095.27 | −3644.63 | −71.53 | | YES | 101.8 |
| 52 | 7135.70 | 6820.16 | −315.54 | −4.63 | | | 102.2 |
| 53 | 41846.79 | 34085.00 | −7761.79 | −22.77 | | YES | 101.8 |
| 54 | 4056.35 | 5380.55 | 1324.20 | 24.61 | YES | | 92.56 |
| 55 | 4862.51 | 4208.02 | −654.50 | −15.55 | | | 87.57 |
| 56 | 6388.90 | 6439.21 | 50.31 | 0.78 | | | 38.45 |
| 57 | 3202.22 | 3420.06 | 217.84 | 6.37 | | | 28.29 |
| 58 | 5089.32 | 5465.22 | 375.90 | 6.88 | | | 28.97 |
| 59 | 4630.56 | 4582.51 | −48.05 | −1.05 | | | 14.51 |
| 60 | 3312.85 | 4716.69 | 1403.84 | 29.76 | YES | | 17.87 |
| 61 | 116209.95 | 99582.33 | −16627.62 | −16.70 | | | 16.68 |
| 62 | 10510.80 | 12492.36 | 1981.56 | 15.86 | | | 15.4 |
| 63 | 270279.41 | 26349.96 | −243929.45 | −925.73 | | YES | 329.7 |
| 64 | 395076.54 | 74767.36 | −320309.18 | −428.41 | | YES | 343.6 |
| 65 | 13595.96 | 12868.71 | −727.24 | −5.65 | | | 342.8 |
| 66 | 13604.39 | 9842.37 | −3762.02 | −38.22 | | YES | 339.9 |
| 67 | 14709.63 | 14402.19 | −307.44 | −2.13 | | | 339.8 |
| 68 | 9689.37 | 11366.29 | 1676.91 | 14.75 | | | 338.6 |
| 69 | 35616.98 | 25804.20 | −9812.78 | −38.03 | | YES | 337.3 |
| 70 | 2983.26 | 5467.90 | 2484.64 | 45.44 | YES | | 339.8 |
| 71 | 3167.16 | 5132.26 | 1965.10 | 38.29 | YES | | 155.3 |
| 72 | 5069.51 | 4752.00 | −317.51 | −6.68 | | | 31.01 |
| 73 | 4072.68 | 5770.29 | 1697.61 | 29.42 | YES | | 32.38 |
| 74 | 5044.98 | 10125.29 | 5080.31 | 50.17 | YES | | 30.41 |
| 75 | 4630.60 | 6491.46 | 1860.86 | 28.67 | YES | | 29.67 |
| 76 | 5965.16 | 7134.39 | 1169.23 | 16.39 | | | 29.63 |
| 77 | 5382.72 | 12500.80 | 7118.08 | 56.94 | YES | | 20.46 |
| 78 | 9035.65 | 12922.74 | 3887.09 | 30.08 | YES | | 19.76 |
| 79 | 8406.07 | 11128.31 | 2722.24 | 24.46 | YES | | 19.58 |
| 80 | 9062.78 | 11407.92 | 2345.14 | 20.56 | YES | | 14.17 |

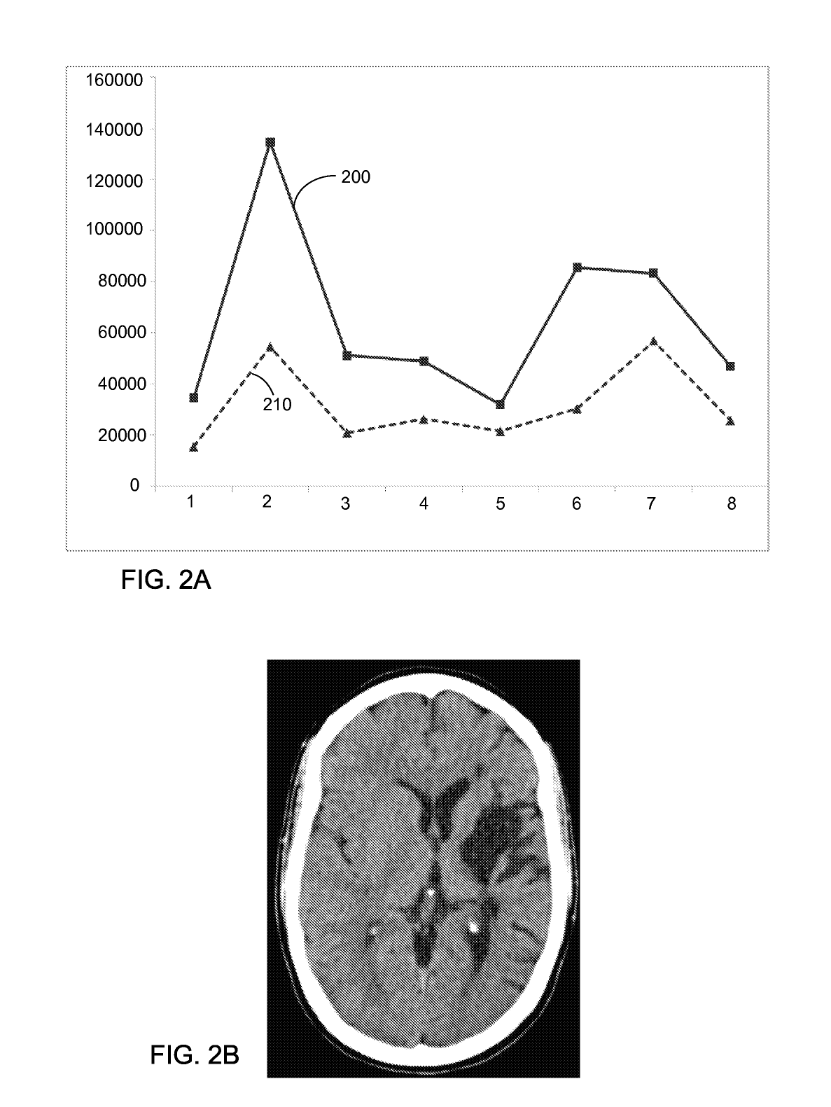

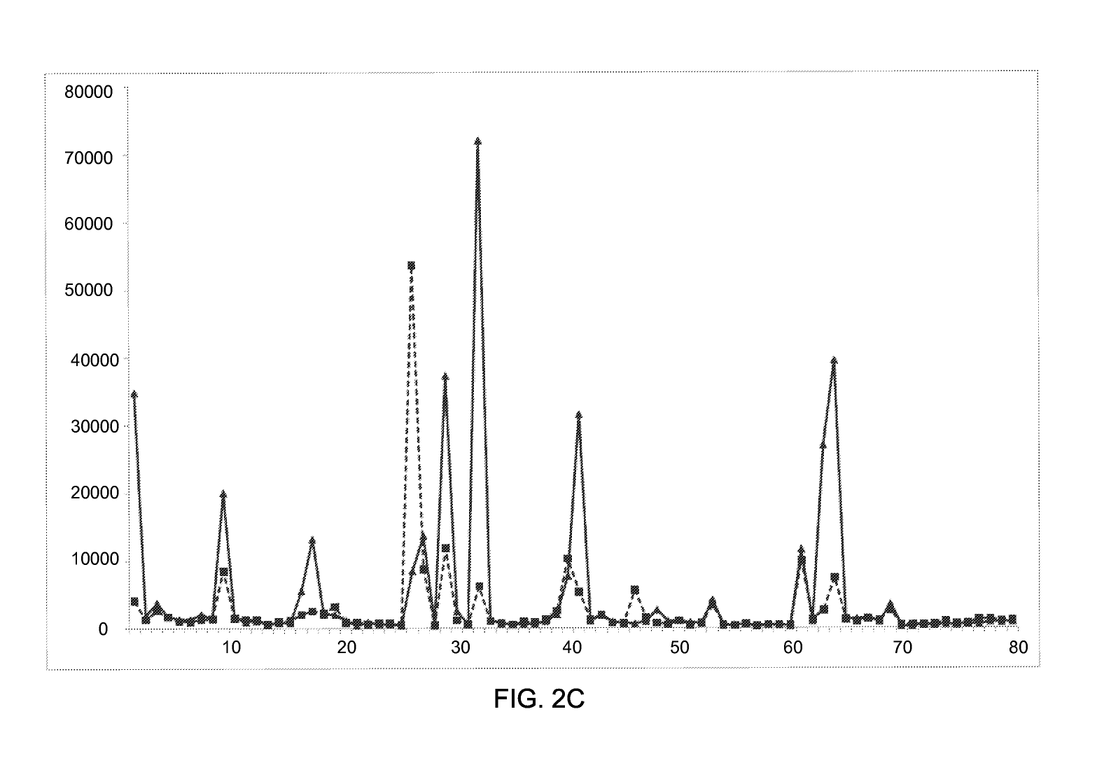

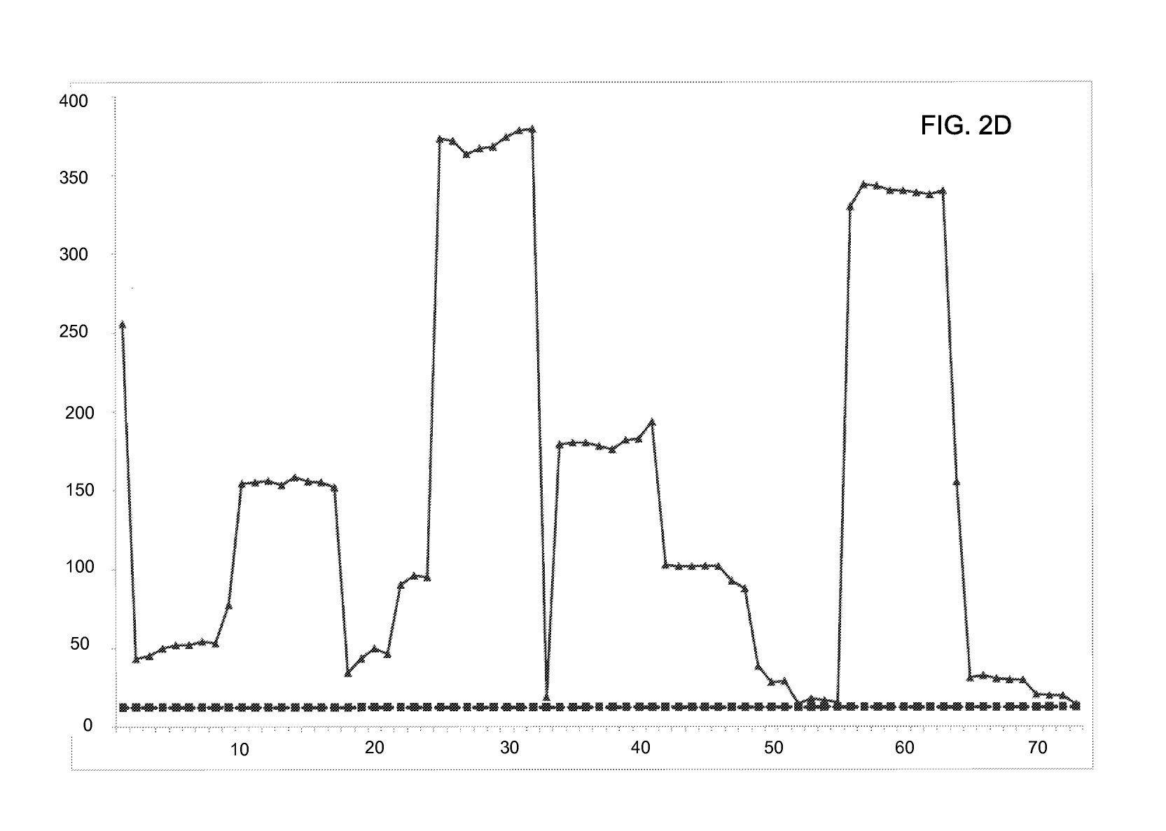

Reference is now made to FIGS. 2A-2D. FIG. 2A illustrates a plot 200 of fronto-temporal activity in the right hemisphere, and a plot 210 of fronto-temporal activity in the left hemisphere of a patient exhibiting a CVA in the left cerebral hemisphere over 8 minutes of sleep, where the x-axis represents time and the y-axis represents EEG signal amplitude. FIG. 2B illustrates a cranial CT scan of the patient of FIG. 2A. FIG. 2C illustrates a plot 220 of fronto-temporal activity in the right hemisphere, and a plot 230 of fronto-temporal activity in the left hemisphere of a healthy individual over 80 minutes of sleep, where the x-axis represents time and the y-axis represents EEG signal amplitude. FIG. 2D illustrates a plot 240 of the values of parameter C1 for the patient of FIG. 2A and a plot 250 of the values of parameter C1 for the healthy individual of FIG. 2C, where the x-axis represents time and the y-axis represents standard deviation values. Table 1 illustrates the calculations of signals Left and Right, the difference there between, the ratio thereof, the standard deviation thereof and the comparison to the respective threshold, for the patient of FIG. 2A. Table 2 illustrates the calculations of signals Left and Right, the difference there between, the ratio thereof, the standard deviation thereof and the comparison to the respective threshold, for the healthy individual of FIG. 2C.

As can be seen from FIGS. 2A-2D and Tables 1 and 2, the EEG data of fronto-temporal activity in a healthy individual, fails to show an at least 20% exceedance in the amplitude of the EEG signal in one hemisphere over the counterpart hemisphere successively over more than 4 timeframes of 1 min, during 80 min of sleep; whereas the EEG data from a patient exhibiting a CVA shows a consistent more than 20% exceedance in the amplitude of the EEG signal from the right hemisphere over the EEG signal from the left hemisphere, successively over 8 timeframes of 1 min. Accordingly, the threshold for parameter Y1 was exceeded in the CVA patient but not in the healthy control individual.

Noticeably, even if the threshold for parameter Y1 was defined as at least 20% exceedance in the amplitude of the fronto-temporal EEG signal in one hemisphere comparatively to the counterpart hemisphere, successively over 5 timeframes of 1 min, in lieu of 8 timeframes of 1 min, such a threshold still would have been exceeded in the CVA patient but not in the healthy control individual.

Moreover, the EEG data of fronto-temporal activity in the CVA patient shows the standard deviation (STDV) of 8 values of parameter Y1, obtained over 73 consecutive blocks of 8 timeframes of 1 min, consistently exceed the numeral value of 13; whereas the EEG data from the healthy individual fails to show an exceedance of the numeral value of 13. Accordingly, the threshold for parameter C1 was exceeded in CVA patient but not in the healthy control individual. Ultimately, the cranial CT scan, shown in FIG. 2B, evidences the occurrence of the CVA at the left fronto-temporal lobe.

Thus, utilizing the apparatus and methods described herein, incidence of CVA may be detected during sleep and appropriate medical personal alerted.

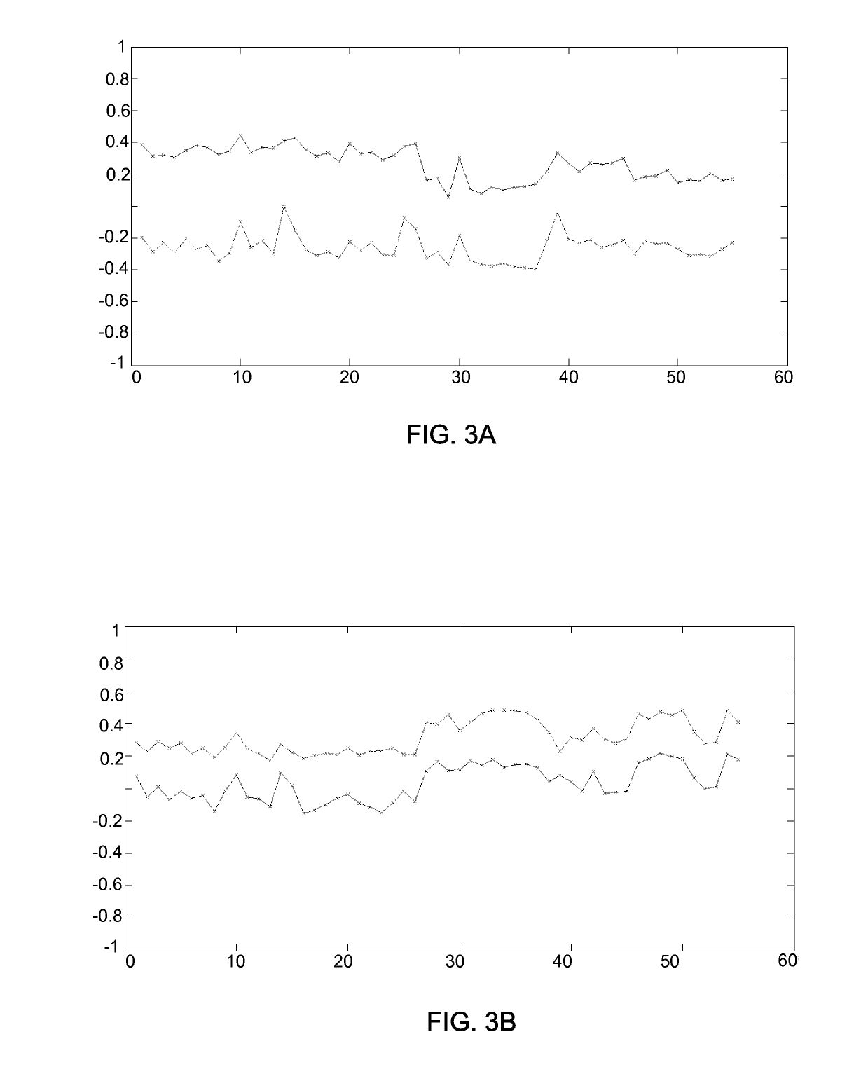

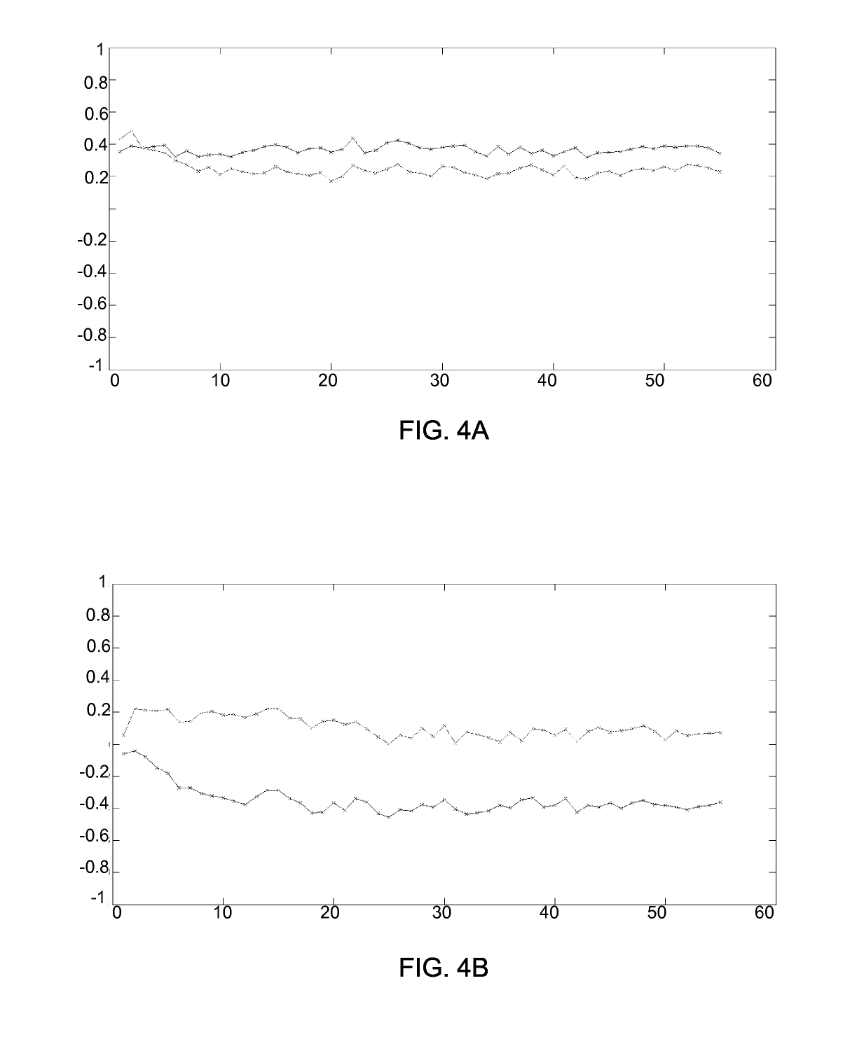

FIGS. 3A-4B illustrate various graphs of the correlation between EEG signals of a plurality of electrodes, where the x-axis denotes sets of EEG samples and the y-axis denotes the correlation between the delta frequency component of EEG signals of two electrodes, the EEG signals sampled from a plurality of patients suffering from CVA.

In particular, FIGS. 3A and 4A each illustrate a plot of the correlation between EEG signals received from electrodes positioned at the F3 and F4 positions, and further illustrates a plot of the correlation between the delta frequency component of EEG signals received from electrodes positioned at the C3 and C4 positions, the EEG signals sampled from a first, second, third, fourth, fifth, sixth and seventh patient, respectively. FIGS. 3B and 4B each illustrate a plot of the correlation between EEG signals received from electrodes positioned at the P3 and P4 positions, and further illustrates a plot of the correlation between the delta frequency component of EEG signals received from electrodes positioned at the O1 and O2 positions, the EEG signals sampled from the first, second, third, fourth, fifth, sixth and seventh patient, respectively.

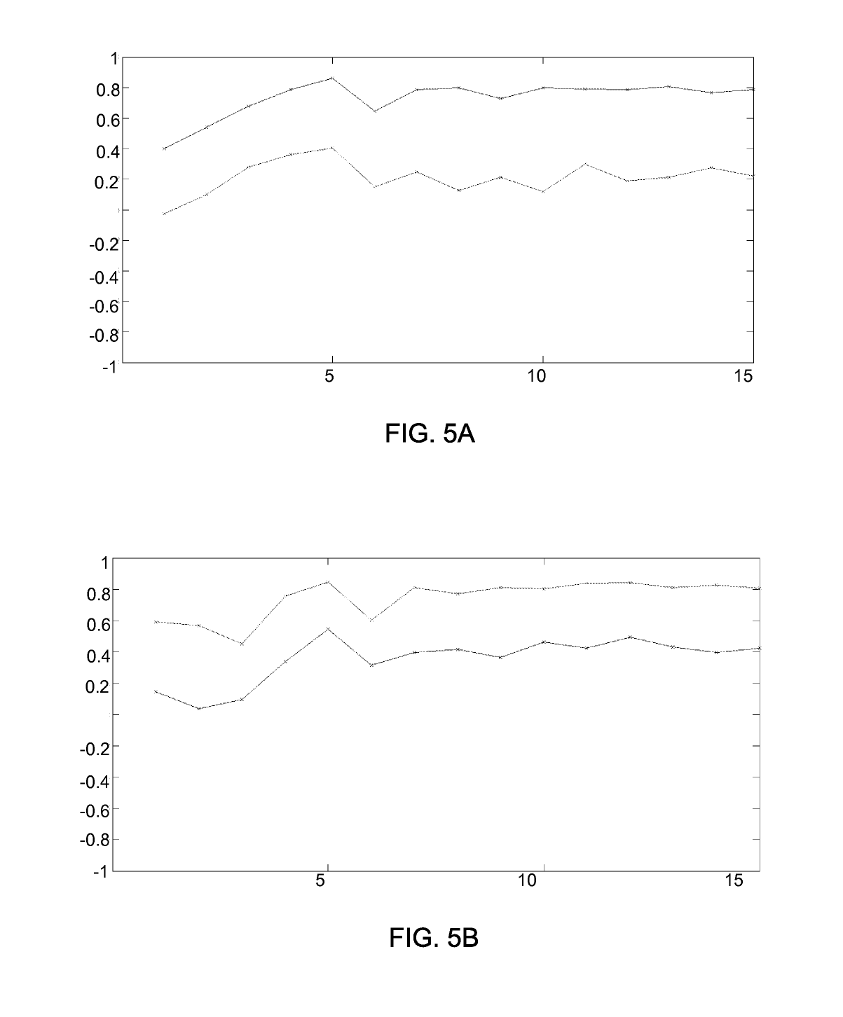

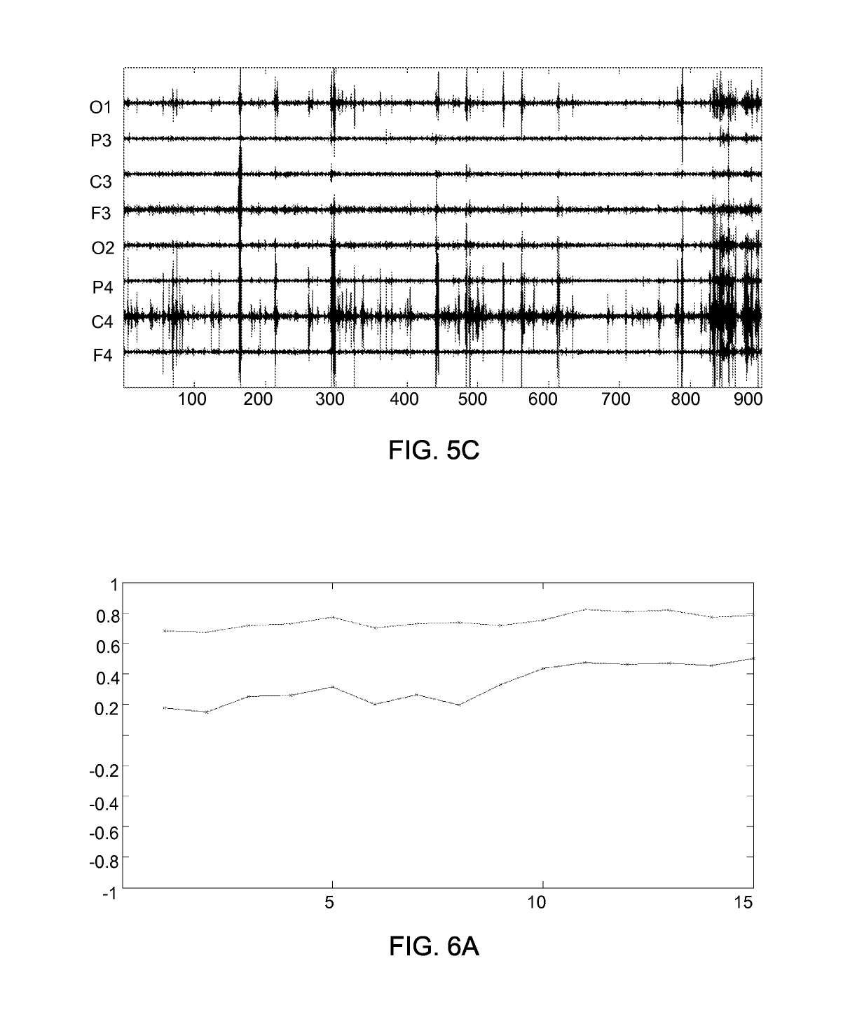

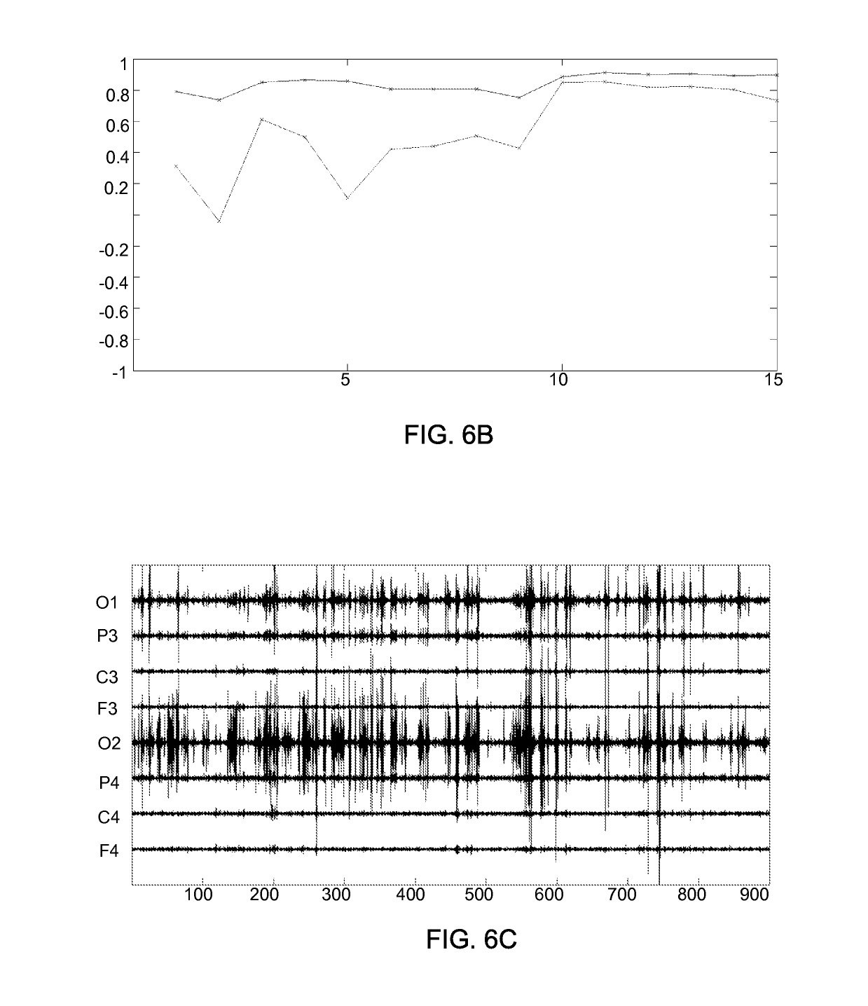

FIGS. 5A and 6A each illustrate a plot of the correlation between EEG signals received from electrodes positioned at the F3 and F4 positions, and further illustrates a plot of the correlation between the delta frequency component of EEG signals received from electrodes positioned at the C3 and C4 positions, the EEG signals sampled from a first and second healthy individual, respectively. FIGS. 5B and 6B each illustrate a plot of the correlation between EEG signals received from electrodes positioned at the P3 and P4 positions, and further illustrates a plot of the correlation between the delta frequency component of EEG signals received from electrodes positioned at the O1 and O2 positions, the EEG signals sampled from the first and second healthy individual, respectively. FIGS. 5C and 6C each illustrate the received EEG signals from a plurality of electrodes sampled from the first and second healthy individual.

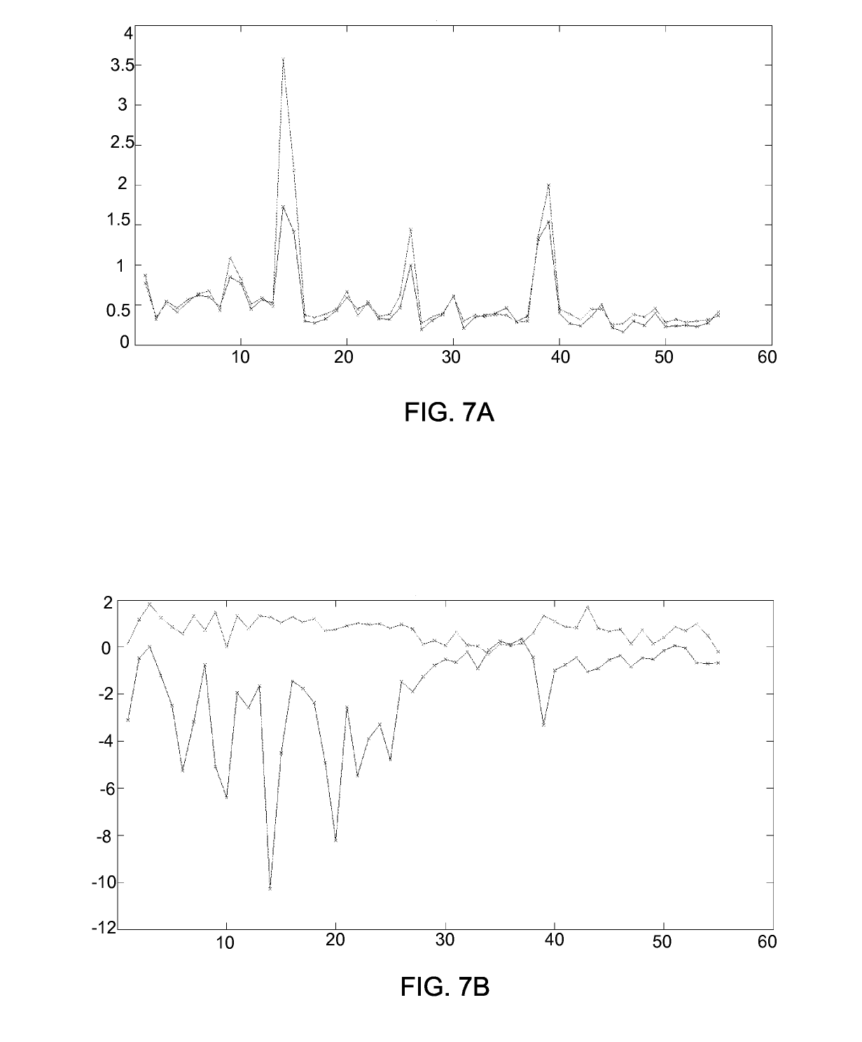

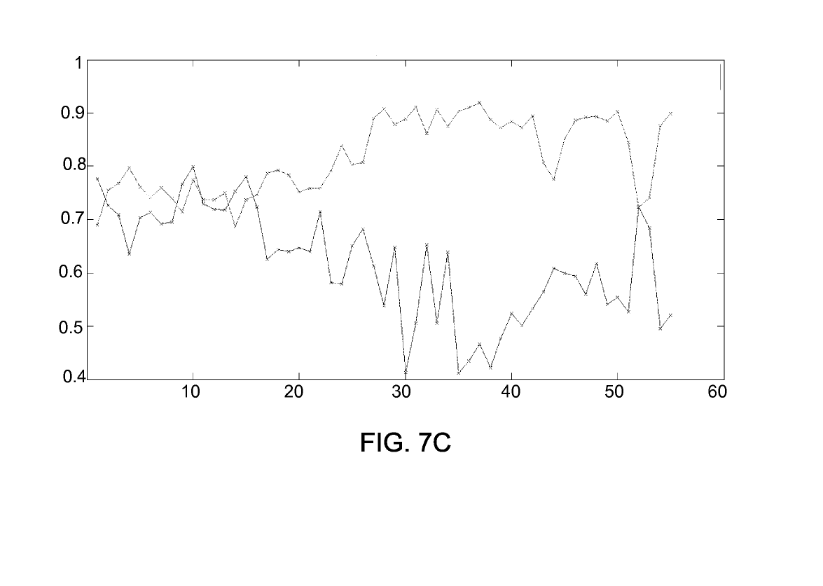

FIG. 7A illustrates a plot of the amplitudes of the alpha frequency component of an EEG signal received from an electrode positioned in the F3 position, and further illustrates a plot of the amplitudes of the alpha frequency component of an EEG signal received from an electrode positioned in the F4 position. FIG. 7B illustrates a plot of the difference between the amplitudes of the alpha frequency component of an EEG signal received from an electrode positioned in the F3 position and the amplitudes of the alpha frequency component of an EEG signal received from an electrode positioned in the C3 position. FIG. 7B further illustrates a plot of the difference between the amplitudes of the alpha frequency component of an EEG signal received from an electrode positioned in the F4 position and the amplitudes of the alpha frequency component of an EEG signal received from an electrode positioned in the C4 position. FIG. 7C illustrates a plot of the correlation between the alpha frequency component of an EEG signal received from an electrode positioned in the F3 position and the alpha frequency component of an EEG signal received from an electrode positioned in the C3 position. FIG. 7C further illustrates a plot of the correlation between the alpha frequency component of an EEG signal received from an electrode positioned in the F4 position and the alpha frequency component of an EEG signal received from an electrode positioned in the C4 position. As illustrated, the amplitude differences of FIG. 7B provides more information than the amplitudes of FIG. 7A. Additionally, at certain time periods, the correlations of FIG. 7C provide more information than the amplitude differences of FIG. 7B.

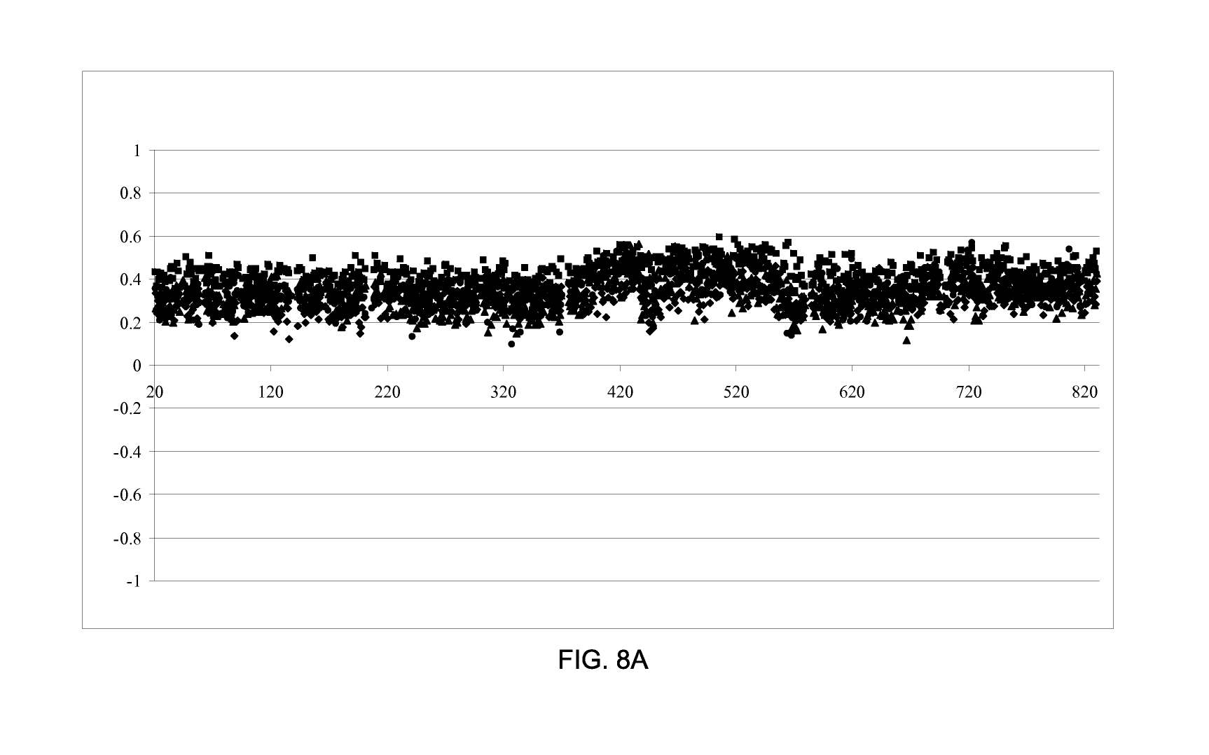

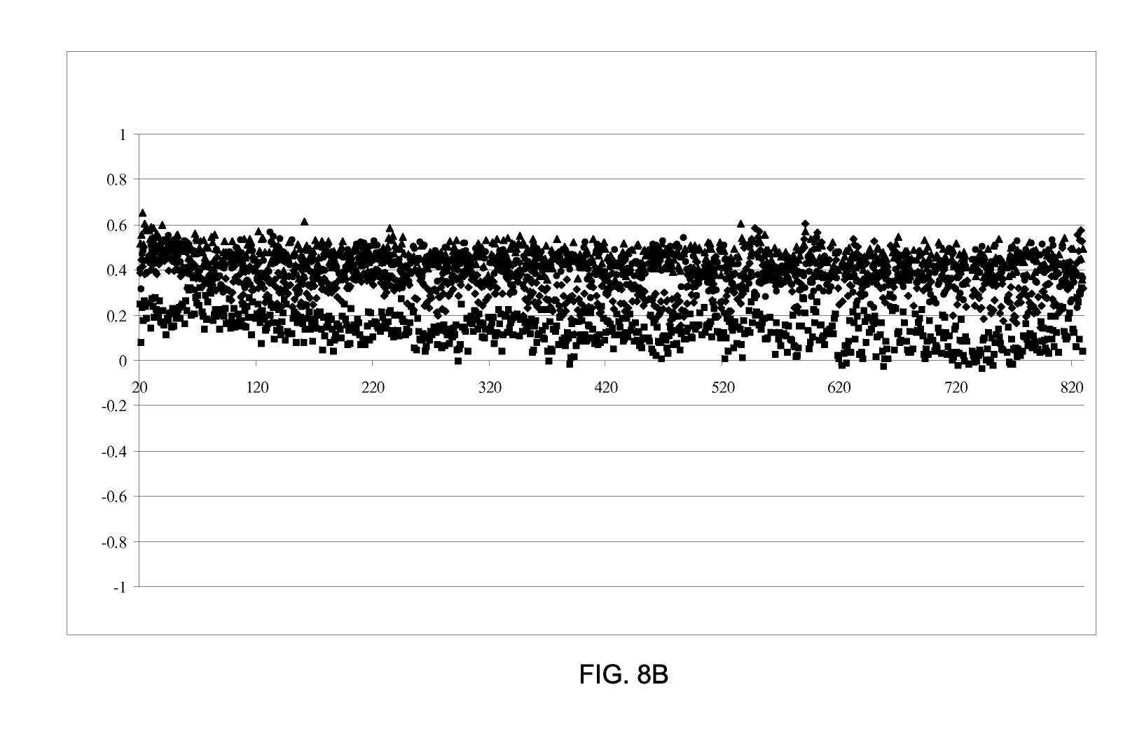

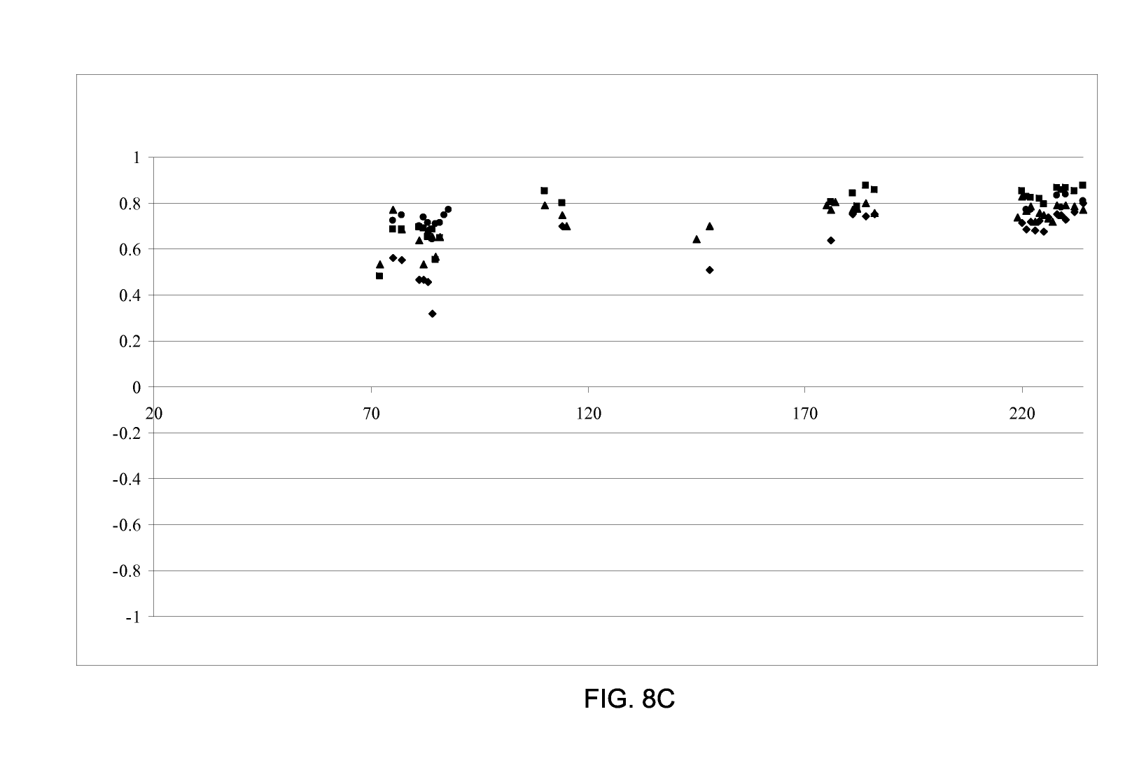

FIG. 8A illustrates correlations between the differences of EEG signals received from the F3 and C3 electrodes of a third patient suffering from CVA and the differences of EEG signals received from the F4 and C4 electrodes of the third patient, the correlations being determined over a plurality of time intervals and denoted with a triangle shape, wherein the x-axis denotes clusters of EEG samples and the y-axis denotes correlation units. At times where noise was detected, no information is displayed. FIG. 8A further illustrates correlations between the differences of EEG signals received from the C3 and P3 electrodes of the third patient and the differences of EEG signals received from the C4 and P4 electrodes of the third patient, the correlations being determined over a plurality of time intervals and denoted with a square shape. FIG. 8A further illustrates correlations between the differences of EEG signals received from the P3 and O1 electrodes of the third patient and the differences of EEG signals received from the P4 and O2 electrodes of the third patient, the correlations being determined over a plurality of time intervals and denoted with a circle shape. FIG. 8A further illustrates correlations between the differences of EEG signals received from the C3 and T3 electrodes of the third patient and the differences of EEG signals received from the C4 and T4 electrodes of the third patient, the correlations being determined over a plurality of time intervals and denoted with a diamond shape.

FIG. 8B illustrates correlations between the differences of EEG signals received from the F3 and C3 electrodes of a fourth patient suffering from CVA and the differences of EEG signals received from the F4 and C4 electrodes of the fourth patient, the correlations being determined over a plurality of time intervals and denoted with a triangle shape, wherein the x-axis denotes clusters of EEG samples and the y-axis denotes correlation units. At times where noise was detected, no information is displayed. FIG. 8B further illustrates correlations between the differences of EEG signals received from the C3 and P3 electrodes of the fourth patient and the differences of EEG signals received from the C4 and P4 electrodes of the fourth patient, the correlations being determined over a plurality of time intervals and denoted with a square shape. FIG. 8B further illustrates correlations between the differences of EEG signals received from the P3 and O1 electrodes of the fourth patient and the differences of EEG signals received from the P4 and O2 electrodes of the fourth patient, the correlations being determined over a plurality of time intervals and denoted with a circle shape. FIG. 8B further illustrates correlations between the differences of EEG signals received from the C3 and T3 electrodes of the fourth patient and the differences of EEG signals received from the C4 and T4 electrodes of the fourth patient, the correlations being determined over a plurality of time intervals and denoted with a diamond shape.

FIG. 8C illustrates correlations between the differences of EEG signals received from the F3 and C3 electrodes of a third healthy individual and the differences of EEG signals received from the F4 and C4 electrodes of the third healthy individual, the correlations being determined over a plurality of time intervals and denoted with a triangle shape, wherein the x-axis denotes clusters of EEG samples and the y-axis denotes correlation units. At times where noise was detected, no information is displayed. FIG. 8C further illustrates correlations between the differences of EEG signals received from the C3 and P3 electrodes of the third healthy individual and the differences of EEG signals received from the C4 and P4 electrodes of the third healthy individual, the correlations being determined over a plurality of time intervals and denoted with a square shape. FIG. 8C further illustrates correlations between the differences of EEG signals received from the P3 and O1 electrodes of the third healthy individual and the differences of EEG signals received from the P4 and O2 electrodes of the third healthy individual, the correlations being determined over a plurality of time intervals and denoted with a circle shape. FIG. 8C further illustrates correlations between the differences of EEG signals received from the C3 and T3 electrodes of the third healthy individual and the differences of EEG signals received from the C4 and T4 electrodes of the third healthy individual, the correlations being determined over a plurality of time intervals and denoted with a diamond shape.

As shown by FIGS. 8A-8C, the correlation between electrodes in opposing cerebral hemispheres in patients suffering from CVA is generally lower than the correlation between electrodes in a healthy individual.

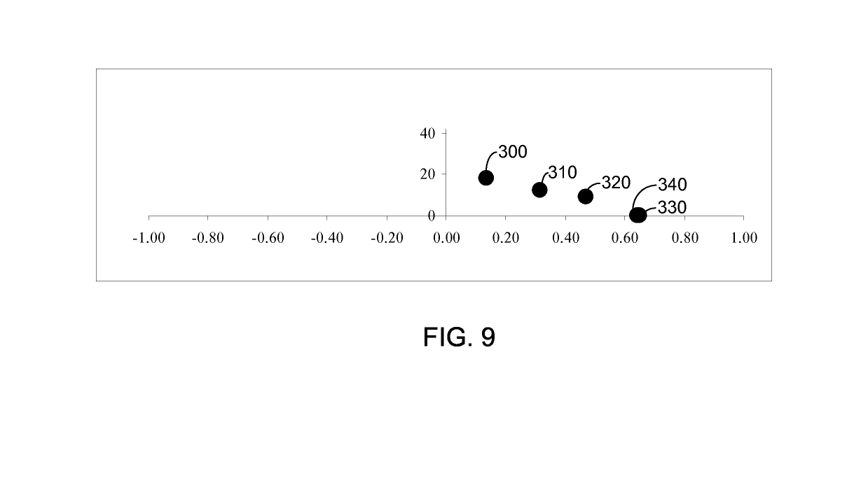

FIG. 9 illustrates a graph of the lowest correlation average measured between pairs of electrodes positioned in opposing cerebral hemispheres, wherein the x-axis denotes correlation units and the y-axis denotes the measured severity of the CVA for each patient, according to the National Institutes of Health Stroke Scale (NIHSS). In particular, dot 300 illustrates the lowest correlation average between pairs of electrodes for a fifth patient, dot 310 illustrates the lowest correlation average between pairs of electrodes for a sixth patient, dot 320 illustrates the lowest correlation average between pairs of electrodes for a seventh patient, dot 330 illustrates the lowest correlation average between pairs of electrodes for a fourth healthy individual and dot 340 illustrates the lowest correlation between pairs of electrodes for a fifth healthy individual. The lowest correlation average is the average of correlations between pairs of electrodes over a plurality of EEG sample clusters, as illustrated in FIGS. 8A-8C, exhibiting the lowest value.

As shown in FIG. 9, as the severity of the CVA increases the correlation between the pairs of electrodes decreases. The actual information of dots 300-340 are described in Table 3, which includes: the value of the lowest correlation average for the particular patient or healthy individual; the severity of each CVA; and the location of each CVA, i.e. cortical, sub-cortical, or both:

TABLE 3

| | | | Cortical/ |
|---|---|---|---|
| | Lowest Average | NIHSS | sub-cortical |
| Healthy 4 | 0.65 | 0 | |
| Healthy 5 | 0.65 | 0 | |
| Patient 5 | 0.32 | 12 | Cortical |
| Patient 6 | 0.13 | 18 | Both |
| Patient 7 | 0.47 | 9 | Sub-cortical |

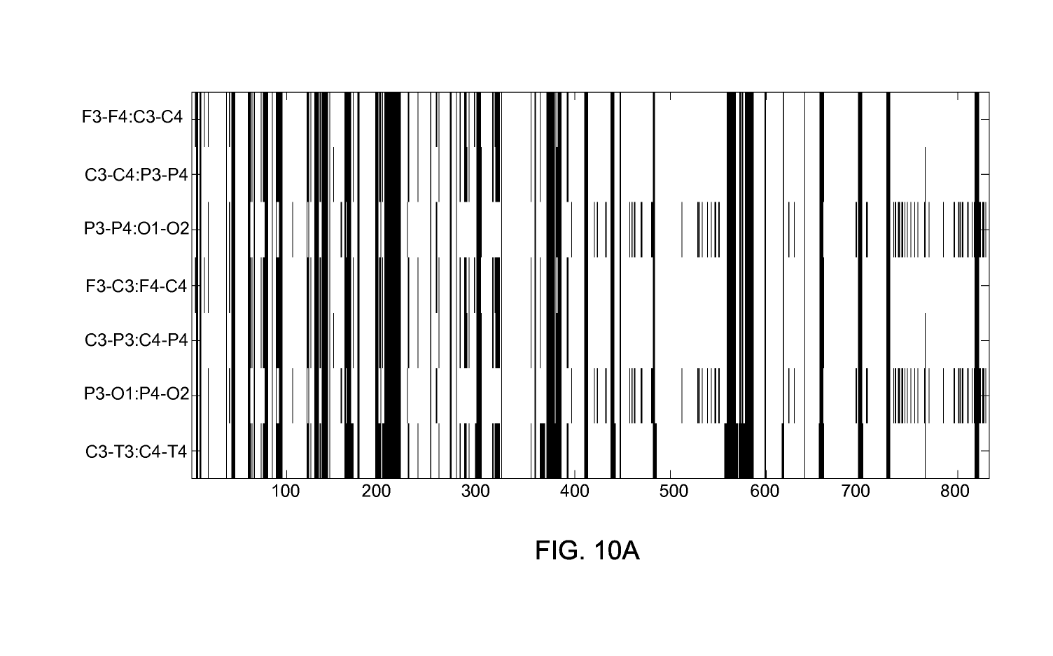

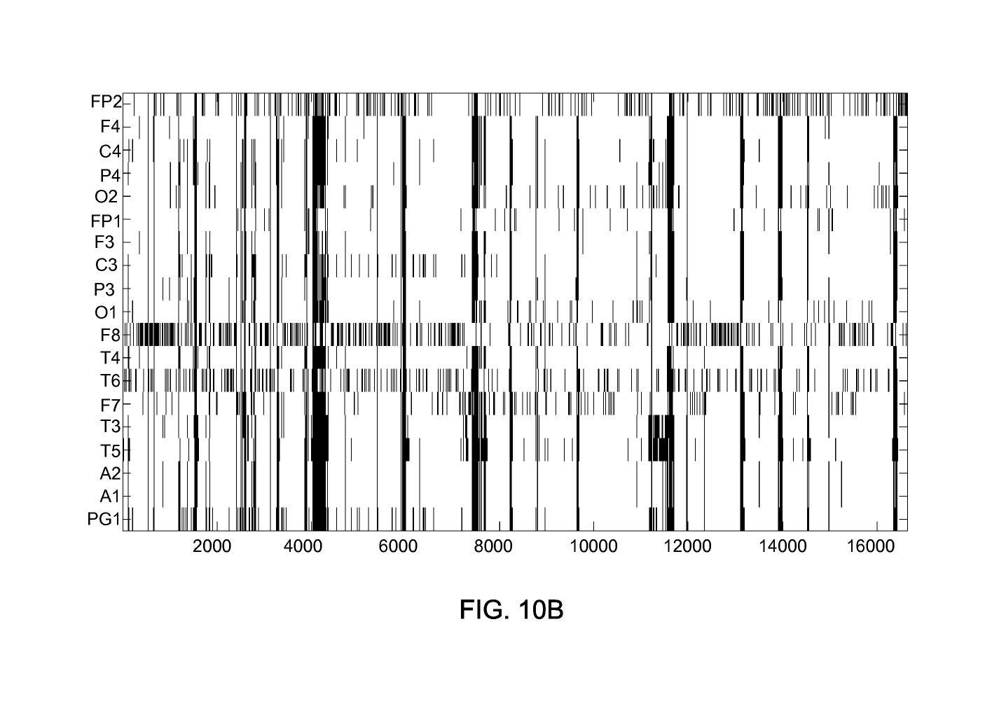

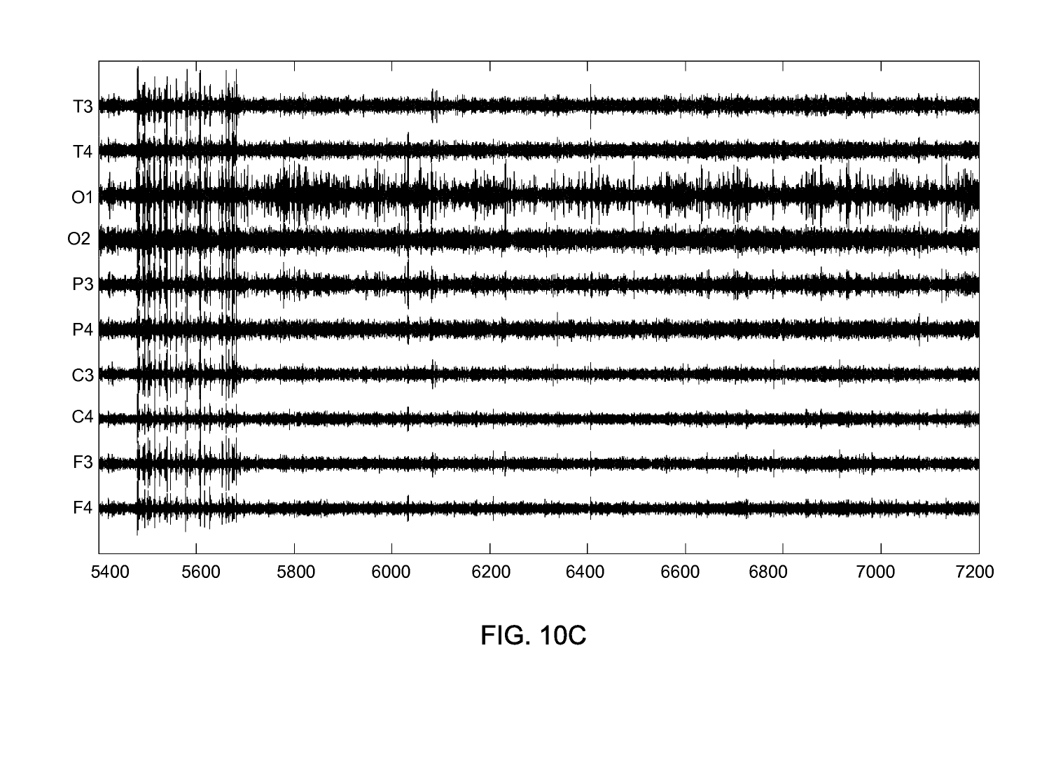

FIG. 10A illustrates a graph of clusters of EEG samples from sets of electrodes which exhibit noise, i.e. exhibit a deviation from the average correlation between EEG signals of pairs of electrodes. The clusters are clusters of EEG signals sampled over 3 seconds. In particular, noisy clusters of EEG samples are illustrated for the sets consisting of: pairs F3-F4 and C3-C4; pairs C3-C4 and P3-P4; pairs P3-P4 and O1-O2; pairs F3-C3 and F4-C4; pairs C3-P3 and C4-P4; pairs P3-O1 and P4-O2; and pairs C3-T3 and C4-T4, wherein the x-axis denotes EEG sample clusters and the y-axis denotes the respective sets of electrodes.

FIG. 10B illustrates a graph of EEG samples from a plurality of electrodes which exhibit noise, i.e. exhibit a deviation from the average EEG activity, wherein the x-axis denotes time in seconds and the y-axis denotes the particular electrodes, in particular electrodes positioned in the Fp2, F4, C4, P4, O2, Fp1, F3, C3, P3, O1, F8, T4, T6, F7, T3, T5, A2, A1 and PG1 positions.

FIG. 10C illustrates the EEG signals received from a portion of the electrodes of FIG. 10B over a portion of the time. In particular, the EEG signals were received from the electrodes positioned in the T3, T4, O1, O2, P3, P4, C3, C4, F3 and F4 positions over a 2000 second interval.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. In the claims of this application and in the description of the invention, except where the context requires otherwise due to express language or necessary implication, the word “comprise” or variations such as “comprises” or “comprising” is used in any inclusive sense, i.e. to specify the presence of the stated features but not to preclude the presence or addition of further features in various embodiments of the invention.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting. No admission is made that any reference constitutes prior art. The discussion of the reference states what their author's assert, and the applicants reserve the right to challenge the accuracy and pertinency of the cited documents. It will be clearly understood that, although a number of prior art complications are referred to herein, this reference does not constitute an admission that any of these documents forms part of the common general knowledge in the art in any country.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. A system for detecting a neurological deterioration in a patient, the system comprising:
an analyzing module; and
at least one of input port arranged to receive a plurality of first samples of electroencephalographic (EEG) activity of the patient and a plurality of second samples of EEG activity of the patient and further arranged to output said received samples to said analyzing module,
wherein said analyzing module is arranged to:
compare a function of said received first EEG samples to a function of said received second EEG samples;
determine the difference between the outcome of said comparison and a baseline value; and
output a neurological deterioration signal responsive to said determined difference.

2. The system of claim 1, wherein said received at least one first EEG sample is associated with a first cerebral hemisphere of the patient and said second plurality of received EEG samples are associated with a second cerebral hemisphere of the patient opposing the first cerebral hemisphere.

3. The system of claim 1, wherein said comparison comprises determining the correlation between the function of said first plurality of received EEG samples and the function of said second plurality of received EEG samples.

4. The system of claim 1, wherein said at least one input port comprises a plurality of input ports, each in communication with a particular electrode arranged to sample EEG samples of the patient, and
wherein said received plurality of first EEG samples comprise:
a first set of first EEG samples received from a first of a pair of first electrodes; and
a second set of first EEG samples received from a second of a pair of first electrodes,
wherein said received plurality of second EEG samples comprise:
a first set of second EEG samples received from a first of a pair of second electrodes; and
a second set of second EEG samples received from a second of a pair of second electrodes,
wherein said function of said received plurality of first EEG samples comprises the difference between the amplitudes of said first set of first EEG samples and the amplitudes of said second set of first EEG samples, and
wherein said function of said received plurality of second EEG sample comprises the difference between the amplitudes of said first set of second EEG samples and the amplitudes of said second set of second EEG samples.

5. The system of claim 1, wherein said at least one input port comprises a plurality of input ports, each in communication with a particular electrode arranged to sample EEG samples of the patient,
wherein said received plurality of first EEG samples comprise:
a first set of first EEG samples received from a first of a pair of first electrodes; and
a second set of first EEG samples received from a second of a pair of first electrodes,
wherein said received plurality of second EEG samples comprise:
a first set of second EEG samples received from a first of a pair of second electrodes; and
a second set of second EEG samples received from a second of a pair of second electrodes,
wherein said function of said received plurality of first EEG samples comprises the correlation between said first set of first EEG samples and said second set of first EEG samples, and
wherein said function of said received plurality of second EEG samples comprises the correlation between said first plurality of second EEG samples and said second plurality of second EEG samples.

6. The system of claim 1, wherein said comparison comprises determining one of:
an average of the amplitude values of said plurality of first EEG samples and said plurality of second EEG samples;
the variability of the amplitude values of said plurality of first EEG samples and said plurality of second EEG samples; and
the rate of change of the amplitude values of said plurality of first EEG samples and said plurality of second EEG samples.

7. The system of claim 1, wherein said baseline value comprises one of:
a general population baseline; and
a baseline associated with the patient.

8. The system of claim 1, further comprising:
a patient stimulation device, in communication with said analyzing module, and arranged, responsive to a non-stimulated state patient stimulation signal, to provide a non-awakening stimulation to the patient, said non-awakening stimulation arranged to at least partially increase the EEG activity of the patient,
wherein said analyzing module is further arranged to:
compare the amplitude of each of said received EEG samples to a predetermined minimum value; and
in the event the amplitude of one of said received EEG samples is less than the predetermined minimum value, output said patient stimulation signal.

9. The system of claim 8, wherein said patient stimulation device is arranged to provide awakening stimulation to the patient responsive to a stimulated state patient stimulation signal received from said analyzing module, and
wherein said awakening stimulation is arranged to awaken the patient.

10. The system of claim 1, further comprising:
a patient stimulation device, in communication with said analyzing module, and arranged, responsive to a non-stimulated state patient stimulation signal, to provide a non-awakening stimulation to the patient,
wherein said non-awakening stimulation is arranged to at least partially increase the EEG activity of the patient, and
wherein said non-stimulated state patient stimulation signal is responsive to said output neurological deterioration signal.

11. The system of claim 10, wherein said patient stimulation device is arranged to provide awakening stimulation to the patient responsive to an stimulated state patient stimulation signal received from said analyzing module,
wherein said awakening stimulation is arranged to awaken the patient, and
wherein said stimulated state patient stimulation signal is responsive to said output neurological deterioration signal.

12. The system of claim 1, wherein said analyzing module further comprises:
a communication module arranged to output said neurological deterioration signal,
wherein said communication module comprises one of:
a connection to a an audible output device;
a connection to a graphical output device;
a connection to a short messaging service;
a connection to a telephony network;
a connection to the Internet; and
a connection to a medical network.

13. The system of claim 1, wherein each of said plurality of input ports is in communication with a particular electrode, said received plurality of first EEG samples being received from at least one first electrode and said received plurality of second EEG samples being received from at least one second electrode,
wherein said analyzing module is further arranged to determine the standard deviation of said received EEG samples from each of the first and second electrodes over a first predetermined time period, and
in the event said determined standard deviation for any the first and second electrodes is greater than a first predetermined value, said analyzing module is further arranged to output a noise detection signal for the particular electrode.

14. The system of claim 1, wherein said neurological deterioration signal is output in the event said determined difference is greater than a second predetermined value for a second predetermined time period.

15. The system of claim 1, wherein said baseline value is selectable from a plurality of baseline values, wherein said analyzing module is further arranged to:
determine the current sleep stage of the patient, responsive to said received plurality of first EEG samples and said plurality of second EEG samples; and
select a particular baseline value responsive to said determined sleep stage of the patient.

16. A method of detecting a neurological deterioration in a patient, the method comprising:
receiving a plurality of first samples of electroencephalographic (EEG) activity of the patient and a plurality of second samples of EEG activity of the patient;
comparing a function of said received first EEG samples to a function of said received second EEG samples;
determining the difference between the outcome of said comparison and a baseline value; and
outputting a neurological deterioration signal responsive to said determined difference.

17. The method of claim 16, wherein said received first EEG samples are associated with a first cerebral hemisphere of the patient and said receive second EEG samples are associated with a second cerebral hemisphere of the patient opposing the first cerebral hemisphere.

18. The method of claim 16, wherein said comparing comprises determining the correlation between the function of said received first EEG samples and the function of said received second EEG samples.

19. The method of claim 16, wherein said received plurality of first EEG samples comprise:
a first set of first EEG samples received from a first of a pair of first electrodes; and
a second set of first EEG samples received from a second of a pair of first electrodes,
wherein said received plurality of second EEG samples comprise:
a first set of second EEG samples received from a first of a pair of second electrodes; and
a second set of second EEG samples received from a second of a pair of second electrodes,
wherein said function of said received plurality of first EEG samples comprises the difference between the amplitudes of said first set of first EEG samples and the amplitudes of said second set of first EEG samples, and
wherein said function of said received plurality of second EEG samples comprises the difference between the amplitudes of said first set of second EEG samples and the amplitudes of said second set of second EEG samples.

20. The method of claim 16, wherein said received plurality of first EEG samples comprise:
a first set of first EEG samples received from a first of a pair of first electrodes; and
a second set of first EEG samples received from a second of a pair of first electrodes,
wherein said received plurality of second EEG samples comprise:
a first set of second EEG samples received from a first of a pair of second electrodes; and
a second set of second EEG samples received from a second of a pair of second electrodes,
wherein said function of said received plurality of first EEG samples comprises the correlation between said first set of first EEG samples and said second set of first EEG samples, and
wherein said function of said received plurality of second EEG samples comprises the correlation between said first set of second EEG samples and said second set of second EEG samples.

21. The method of claim 16, wherein said comparing comprises determining one of:
an average of the amplitude values of said plurality of first EEG samples and said plurality second EEG samples;
the variability of the amplitude values of said plurality of first EEG samples and said plurality of second EEG samples; and
the rate of change of the amplitude values of said plurality of first EEG samples and said plurality of second EEG samples.

22. The method of claim 16, wherein said baseline value comprises one of:
a general population baseline; and
a baseline associated with the patient.

23. The method of claim 16, further comprising:
providing, responsive to a non-stimulated state patient stimulation signal, non-awakening stimulation to the patient, said non-awakening stimulation arranged to at least partially increase the EEG activity of the patient;
comparing the amplitude of each of said received EEG samples to a predetermined minimum value; and
in the event the amplitude of one of said received EEG samples is less than the predetermined minimum value, outputting said patient stimulation signal.

24. The method of claim 23, further comprising:
providing awakening stimulation to the patient responsive to a stimulated state patient stimulation signal, wherein said awakening stimulation is arranged to awaken the patient.

25. The method of claim 16, further comprising:
providing, responsive to a non-stimulated state patient stimulation signal, non-awakening stimulation to the patient,
wherein said non-awakening stimulation is arranged to at least partially increase the EEG activity of the patient, and
wherein said non-stimulated state patient stimulation signal is responsive to said output neurological deterioration signal.

26. The method of claim 25, further comprising:
providing awakening stimulation to the patient responsive to an stimulated state patient stimulation signal,
wherein said awakening stimulation is arranged to awaken the patient, and
wherein said stimulated state patient stimulation signal is responsive to said output neurological deterioration signal.

27. The method of claim 16, wherein said outputting said neurological deterioration signal comprises connecting to one of:
an audible output device;
a graphical output device;
a short messaging service;
a telephony network;
the Internet; and
a medical network.

28. The method of claim 16, wherein said received plurality of first EEG samples are received from at least one first electrode and said received plurality of second EEG samples are received from at least one second electrode, the method further comprising:
determining the standard deviation of said received EEG samples from each of the first and second electrodes over a first predetermined time period, and
in the event said determined standard deviation for any of the first and second electrodes is greater than a first predetermined value, outputting a noise detection signal for the particular electrode.

29. The method of claim 16, wherein said neurological deterioration signal is output in the event said determined difference is greater than a second predetermined value for a second predetermined time period.

30. The method of claim 16, wherein said baseline value is selectable from a plurality of baseline values, the method further comprising:
determining the sleep stage of the patient responsive to said received plurality of first EEG samples and said received plurality of second EEG samples; and
selecting a particular baseline value responsive to said determined sleep stage of the patient.
